(19) United States
(12) Patent Application Publication
(10) Pub. No.: US 2023/0338316 A1
(43) Pub. Date: Oct. 26, 2023

(54) ABBAPOLINS AS INHIBITORS AND DEGRADERS OF POLO-LIKE KINASES

(71) Applicant: UNIVERSITY OF SOUTH CAROLINA, COLUMBIA, SC (US)

(72) Inventors: CAMPBELL MCINNES, COLUMBIA, SC (US); MICHAEL D. WYATT, COLUMBIA, SC (US); DANDA PANI CHAPAGAI, COLUMBIA, SC (US); JESSY M. STAFFORD, CAYCE, SC (US); GURSANKER RAMAMOORTHY, COLUMBIA, SC (US)

(21) Appl. No.: 18/304,048

(22) Filed: Apr. 20, 2023

Related U.S. Application Data
(60) Provisional application No. 63/332,919, filed on Apr. 20, 2022.

(51) Int. Cl. A61K 31/196; A61K 31/662; A61K 31/661

(52) U.S. Cl. CPC A61K 31/196; A61K 31/662; A61K 31/661

(57) ABSTRACT

Disclosed here are methods for treating a cancer, the method comprising administering to a subject in need thereof a therapeutically effective amount of a compound of Formula (I):

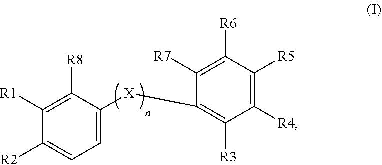

or a pharmaceutically acceptable salt or stereoisomer thereof,

### CROSS REFERENCE TO RELATED APPLICATION

This application claims filing benefit of U.S. Provisional Patent Application Ser. No. 62/332,919, having a filing date of Apr. 20, 2022, which is incorporated herein by reference for all purposes.

### FEDERAL RESEARCH STATEMENT

This invention was made with government support under 1R41CA213711 awarded by the National Institutes of Health. The government has certain rights in the invention.

### BACKGROUND

PLK1, polo-like kinase 1, is a central player regulating mitosis. Inhibition of the sub-cellular localization and kinase activity of PLK1 through the PBD, polo-box domain, is a viable alternative to ATP-competitive inhibitors for which the development of resistance and inhibition of related PLK family members are concerns. The PBD is a phospho-dependent docking motif that is responsible for substrate recognition and subcellular localization.

PLK1 activity has been shown to be a key factor in the development of resistance to current drugs. Having an effective PLK inhibitor will therefore be potentially useful in overcoming this resistance. There are currently no approved drugs based on PLK1 inhibition.

Although PLK1-3 have a high level of sequence identity, studies have shown that each individual PBD has a distinct substrate specificity. Such differences potentially allow for versatility in the development of substrate mimics for target-based therapeutics. Several groups have generated small molecule inhibitors of the PLK1 PBD through high throughput screening approaches. Unfortunately, many of these compounds proved to be non-specific protein alkylators with little potential for further drug development.

Accordingly, alternative approaches and effective small molecule PLK inhibitors are needed in the art. Particularly, highly selective PLK1 inhibitors would be of great benefit in the art.

### SUMMARY

According to one embodiment, disclosed here are methods for treating a cancer, the method comprising administering to a subject in need thereof a therapeutically effective amount of a compound of Formula (I):

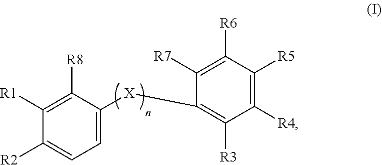

or a pharmaceutically acceptable salt or stereoisomer thereof, wherein:
R1 comprises a hydrogen, —C1 to C8 alkyl, —C1 to C8 alkoxy, or —OH;
R2 is independently selected from hydrogen, (C1 to C8) alkyl, (C1 to C8) alkoxy, secondary (C1 to C8) alkyl methylamine (C8N(Me)), —CONH—(C1 to C6 alkyl), (C1 to C9 alkyl)-sulfinylamino chloride, or NH₂;
R3 through R7 are independently selected from carboxyl, hydrogen, alkyl phosphonate, phosphonate, alkyl phosphonate monoester, phosphate, halogen, alkyl phosphonate disopropylester, phosphate di-t-butylester, —SO₂—F, —OSO₂F, —NSO₂F, di-tert-butyl N,N-diethylphosphoramidite, methyl pivalate, or isobutyl methyl carbonates;
X is carbonyl, sulfonyl, amide, —C1 to C4 alkyl-NH—, —NH—, —C1 to C4 alkyl-S, —SONH—, or —SO2N—;
n is 0, 1, or 2.

### BRIEF DESCRIPTION OF THE DRAWINGS

The present disclosure may be better understood with reference to the drawings, in which:

FIG. 1 illustrates the binding mode of inhibitor SCCP ID No. 6359 with the PBD of PLK1.

FIG. 2 is a table of PLK1 inhibitors as described herein (SCCP ID numbers provided on FIG. 2 are utilized throughout this disclosure).

FIG. 3 graphically illustrates pharmacokinetics of SCCP ID No. 6359 in swiss webster mice at a dosage of 50 mg/kg.

FIG. 4A illustrates the mice's liver weight after treating prostate cancer (PC3) cells with disclosed PLK1 inhibitors.

FIG. 4B illustrates the mice's kidney weight after treating prostate cancer (PC3) cells with disclosed PLK1 inhibitors.

FIG. 4C illustrates the mice's prostate weight following treatment of prostate cancer (PC3) cells with disclosed PLK1 inhibitors.

FIG. 4D illustrates body weight of the mice following treating prostate cancer (PC3) cells with disclosed PLK1 inhibitors.

FIG. 5 illustrates growth curves of PC3 prostate cancer xenografts in athymic nude mice treated with disclosed PLK1 inhibitors compared to phosphate buffer saline (PBS).

FIG. 6 illustrates tumor volume following 15 days of treating PC3 prostate cancer with disclosed PLK1 inhibitors.

FIG. 7A illustrates representative blots of PLK and GADPH detected from isolated xenograft PC3 tumors.

FIG. 7B illustrates percentage of PLK1 detected from isolated xenograft PC3 tumors.

FIG. 8A illustrates degradation of PLK1 by 6369 in PC3 cell line.

FIG. 8B illustrates degradation of PLK1 by 6369 in PC3 cell line after 24 hours of treatment.

FIG. 9A illustrates degradation of PLK1 by 6369 in HeLa cell line.

FIG. 9B illustrates degradation of PLK1 by 6369 in HeLa cell line at various time points.

FIG. 10A illustrates degradation of PLK1 by 6329 in PC3 cell line.

FIG. 10B illustrates degradation of PLK1 by 6329 in PC3 cell line after 24 hours of treatment.

FIG. 11A illustrates degradation of PLK1 by 6329 in HeLa cell line.

FIG. 11B illustrates degradation of PLK1 by 6329 in HeLa cell line at various time points.

FIG. 12 illustrates cell viability assay of 6277 in SH-SY5Y cells.

FIG. 13 illustrates cell viability assay of 6369 in SH-SY5Y cells.

FIG. 14 illustrates degradation of PLK1 by 6369 in SH-HY5Y cells.

FIG. 15 illustrates degradation of PLK1 by 6277 in SH-HY5Y cells.

FIG. 16 illustrates degradation of PLK1 by 6277 in combination with MG132 in SH-HY5Y cells.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to non-peptidic PBD binding inhibitors, termed abbapolins, identified through successful application of the REPLACE strategy that demonstrate potent anti-proliferative activity in prostate tumors and other cell lines. Disclosed, abbapolins show PLK1-specific binding and inhibitory activity as measured by a cellular thermal shift assay and an ability to block phosphorylation of TCTP, a validated target of PLK1-mediated kinase activity. Disclosed abbapolins induce PLK1 degradation in manner that closely matches antiproliferative activity. Moreover, the abbapolins demonstrate antiproliferative activity in cells that are dramatically resistant to ATP-competitive PLK1 inhibitors. Furthermore, the abbapolin derivatives have good oral pharmacokinetics and significant evidence of in vivo antitumor activity. Abbapolins may also serve as a prodrug, which is converted to its active form in vivo.

Polo-like kinase 1 (PLK1) is a central player in regulating entry into and progression through mitosis. Inhibition of sub-cellular localization and kinase activity of PLK1 through the Polo-box domain (PBD) is emerging as a viable alternative to ATP binding site directed drugs for which the development of resistant mutants and inhibition of closely related members of the PLK family (tumor suppressor roles) are primary concerns. I describe related novel non-peptidic PBD binding inhibitors, termed abbapolins, identified through successful application of the REPLACE strategy and demonstrate their potent anti-proliferative activity in prostate tumors and other cell lines. Furthermore, the abbapolins show PLK1-specific binding and inhibitory activity as measured by a cellular thermal denaturation assay and their ability to block phosphorylation of TCTP, a key marker of PLK1 mediated kinase activity. I also made a novel observation that abbapolins upon binding to PLK1 induced its intracellular loss in a mechanism at least partially dependent on the proteasome. The therapeutic potential of these compounds was further indicated through their antiproliferative activity on a cell line (C67V PLK1 mutation) which is dramatically resistant to ATP competitive PLK1 inhibitors.

In some embodiments, the present disclosure is based on the findings that catalytic site binding by BI2536 or volasertib unexpectedly decreased the stability of PLK1 as determined by CETSA, suggesting an induction of a conformational change in intracellular PLK1. In contrast, abbapolins produced the expected right shift in the melting curve of PLK1 indicating binding and stabilization. Intriguingly, these differential effects on PLK1 thermal stability have opposing impacts on the fate of intracellular PLK1. Binding by catalytic inhibitors cause accumulation of PLK1, whereas PBD binding by abbapolins ultimately lead to its loss in cells. Collectively, the results shed further insight into the unique mechanism of action for abbapolins potentially due to their engagement of a cryptic hydrophobic pocket of the PBD. Furthermore, in vivo pharmacokinetic studies showed that optimized abbapolins have promising oral bioavailability and inhibited the growth of prostate tumors in a mouse xenograft experiment. Abbapolins are thus a compelling alternative to catalytic-based inhibitors as the basis for the development of novel therapeutics targeting PLK1.

The following description and other modifications and variations to the presently disclosed subject matter may be practiced by those of ordinary skill in the art, without departing from the spirit and scope of the present disclosure. In addition, it should be understood that aspects of the various embodiments may be interchanged both in whole and in part. Furthermore, those of ordinary skill in the art will appreciate that the following description is by way of example only and is not intended to limit the subject matter.

The present disclosure is generally directed to small molecule PLK inhibitors that can target the polo box domain (PBD). All PLKs contain an N-terminal Ser/Thr kinase catalytic domain and a C-terminal region that includes the PBD. In the absence of a bound substrate, the PBD inhibits the basal activity of the kinase domain. Phosphorylation-dependent binding of the PBD to its ligands releases the kinase domain, while simultaneously localizing polo-like kinases to specific subcellular structures. Thus, the PBD is critical for PLK subcellular localization and substrate recognition prior to phosphorylation.

PLK1 localizes to centrosomes and kinetochores in prophase and to the spindle midzone later in mitosis, which depends on the PBD but not on its kinase activity. For instance, a PBD fragment fused with a membrane-permeable delivery peptide can cause mitotic arrest and cell death in tumor cells. Also, inducible expression of the PLK1 PBD domain fragment in PC-3 prostate cancer cells has been shown to result in significant growth inhibition, validating the concept that interfering with the PBD suppresses the proliferative effects of PLK1. Crystallography has revealed the molecular basis for PLK1 localization through the PBD and peptide inhibitors have been identified. The lack of success of HTS approaches confirms that alternative approaches to PBD inhibitor development are desirable.

The PLK inhibitors disclosed herein are small molecule inhibitors that can target the PBD. As utilized herein, the term “small molecule” refers to a non-peptidic compound that is generally about 1000 Daltons or less (i.e., atomic mass units, one Dalton being equivalent to 1/12 the mass of a ¹²C isotope). In other embodiments, the small molecule inhibitor may be about 500 Daltons or less, about 400 Daltons or less, or about 300 Daltons or less.

Beneficially, the small molecule PBD-targeted inhibitors can retain activity exhibited by peptidic inhibitors, e.g., antitumor activity against cancer cells, and, in one particular embodiment, can exhibit activity against cancer cells that can acquire resistance to ATP-based inhibitors. As such, in one embodiment, disclosed inhibitors can be used in combination with ATP-based inhibitors as a synergistic means of PLK1 targeting in the clinic. Moreover, by targeting non-catalytic functions, PLK can be less likely to obtain resistance to the inhibitors.

The non-peptidic inhibitors have been developed with activity and cellular phenotypes consistent with target engagement of PLK1, and, in one embodiment, can induce apoptosis. As such, the inhibitors can be useful as mechanistic probes to characterize cellular defects of blocking PLK1 independent of catalytic activity, and can be used in combination with catalytic PLK1 inhibitors as a dual approach to attacking PLK1, analogous to many successful clinical precedents (e.g., Bactrim (antibiotic), Combivir® (antiviral), pertuzumab and trastuzumab used in HER2 breast cancer).

Disclosed PBD-targeted inhibitors that are specific for PLK1 are also much less likely to affect the activity of the PLK3 tumor suppressor, as certain of the PLK1 PBD domain inhibitors have minimal activity against PLK3, as discussed further herein.

The small molecule inhibitors can exhibit comparable affinity to peptidic PBD inhibitors and can possess anti-proliferative phenotypes in cells consistent with the observed decrease in PLK1 centrosomal localization. The inhibitors can demonstrate evidence of enhanced PLK1 inhibition in cells relative to peptides and can induce monopolar and multipolar spindles, in contrast to previously reported small molecule PBD inhibitors that display phenotypes only partially representative of PLK1 knockdown. The inhibitors can function as isotype, kinase-selective, non-ATP competitive inhibitors and can be utilized as PLK1 selective anti-tumor therapeutics.

The small molecule inhibitors described herein include an alkyl benzamido benzoic acid core structure that has been utilized to build the PBD inhibitors with high potency and selectivity. As utilized herein, unless otherwise noted, the term “alkyl” refers to any straight-chain or branched, substituted, or unsubstituted C1 to C20 alkyl group.

In one embodiment, a non-peptidic small molecule PLK inhibitor can have the general structure of:

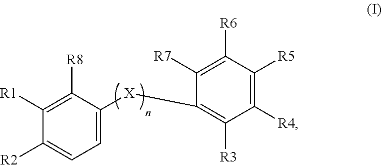

or a pharmaceutically acceptable salt or stereoisomer thereof, wherein:
R1 comprises a hydrogen, —C1 to C8 alkyl, —C1 to C8 alkoxy, or —OH;
R2 is independently selected from hydrogen, (C1 to C8) alkyl, (C1 to C8) alkoxy, secondary (C1 to C8) alkyl methylamine (C8N(Me)), —CONH—(C1 to C6 alkyl), (C1 to C9 alkyl)-sulfinylamino chloride, or NH₂;
R3 through R8 are independently selected from carboxyl, hydrogen, alkyl phosphonate, phosphonate, alkyl phosphonate monoester, phosphate, halogen, alkyl phosphonate disopropylester, phosphate di-t-butylester, —SO₂F, —OSO₂F, —NSO₂F, di-tert-butyl N,N-diethylphosphoramidite, methyl pivalate, or isobutyl methyl carbonates;
X is carbonyl, sulfonyl, amide, —C1 to C4 alkyl-NH—, —NH—, —C1 to C4 alkyl-S, —SONH—, or —SO₂N—;
n is 0, 1, or 2.

In one embodiment, X of Formula (I) may be a carbonyl. For instance, Formula (I) may be represented as:

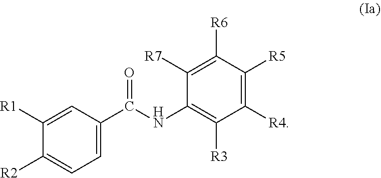

In another embodiment, X of Formula (I) may be a sulfonyl. For instance, Formula (I) may be represented as:

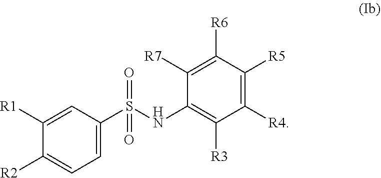

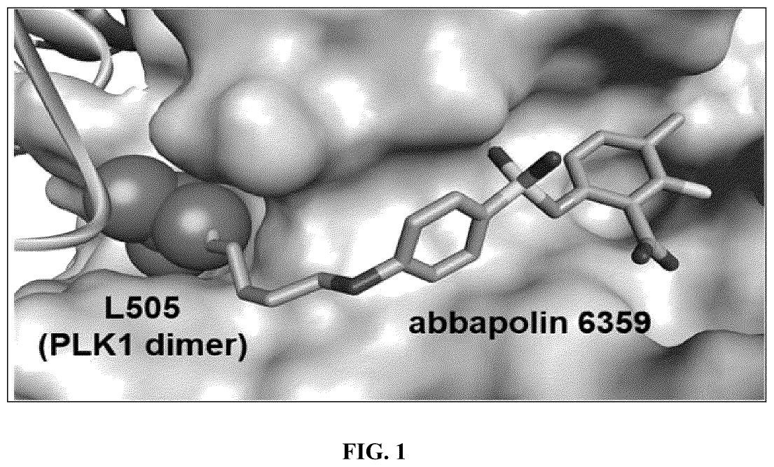

FIG. 1 presents the structure, structure activity relationship (SAR) and interaction data of several representative small molecule PLK inhibitors of this embodiment. A clear SAR for substituents at the 3 or 4 position of increasing alkyl chain length has been established and provides a solid basis and rationale for improvements in potency.

In another embodiment, the small molecule PLK inhibitors can have the following exemplary structure (Table 1):

TABLE 1

| DDBS | |
|---|---|
| ID: | Structure |
| 6153 | 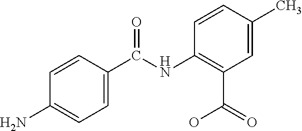 [SMILES: Cc1ccc(NC(=O)c2ccc(N)cc2)c(C(=O)O)c1] |
| 6154 | 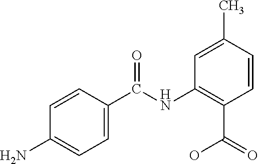 [SMILES: Cc1ccc(C(=O)O)c(NC(=O)c2ccc(N)cc2)c1] |
| 6165 | 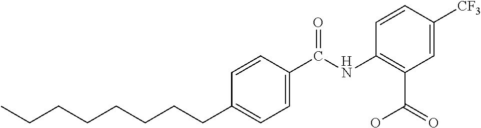 [SMILES: CCCCCCCCc1ccc(C(=O)Nc2ccc(C(F)(F)F)cc2C(=O)O)cc1] |
| 6171 | 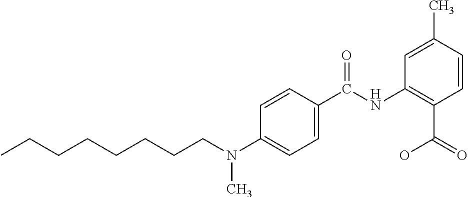 [SMILES: CCCCCCCCN(C)c1ccc(C(=O)Nc2cc(C)ccc2C(=O)O)cc1] |
| 6172 | 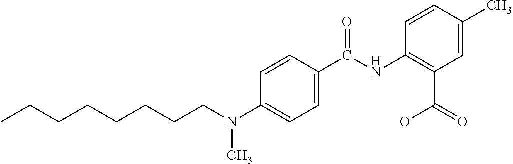 [SMILES: CCCCCCCCN(C)c1ccc(C(=O)Nc2ccc(C)cc2C(=O)O)cc1] |
| 6184 | 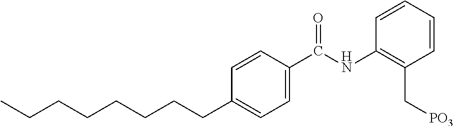 |
| 6189 | 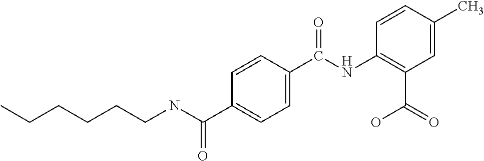 [SMILES: CCCCCCNC(=O)c1ccc(C(=O)Nc2ccc(C)cc2C(=O)O)cc1] |
| 6195 | 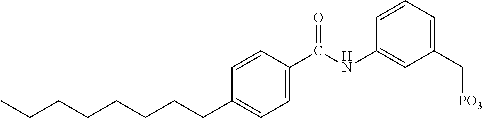 |
| 6237 | 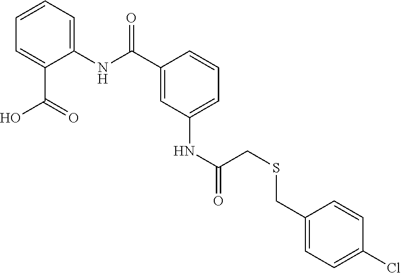 [SMILES: O=C(CSCc1ccc(Cl)cc1)Nc1cccc(C(=O)Nc2ccccc2C(=O)O)c1] |
| 6277 | 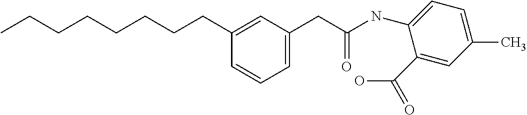 [SMILES: CCCCCCCCc1cccc(CC(=O)Nc2ccc(C)cc2C(=O)O)c1] |
| 6279 | 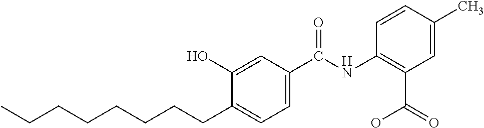 [SMILES: CCCCCCCCc1ccc(C(=O)Nc2ccc(C)cc2C(=O)O)cc1O] |
| 6280 | 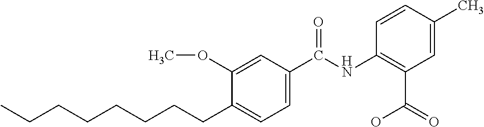 [SMILES: CCCCCCCCc1ccc(C(=O)Nc2ccc(C)cc2C(=O)O)cc1OC] |
| 6281 | 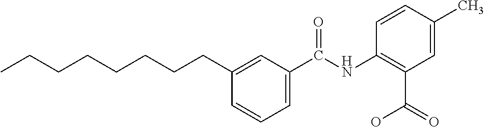 [SMILES: CCCCCCCCc1cccc(C(=O)Nc2ccc(C)cc2C(=O)O)c1] |
| 6282 | 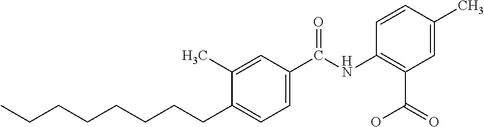 [SMILES: CCCCCCCCc1ccc(C(=O)Nc2ccc(C)cc2C(=O)O)cc1C] |
| 6286 | 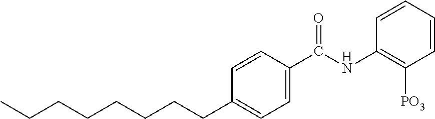 |
| 6287 | 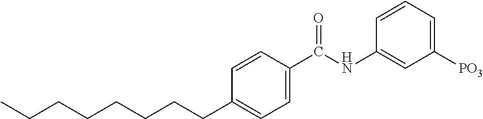 |
| 6355 | 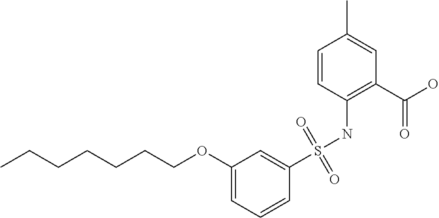 [SMILES: CCCCCCCOc1cccc(S(=O)(=O)Nc2ccc(C)cc2C(=O)O)c1] |
| 6356 | 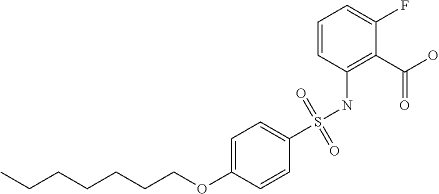 [SMILES: CCCCCCCOc1ccc(S(=O)(=O)Nc2cccc(F)c2C(=O)O)cc1] |
| 6358 | 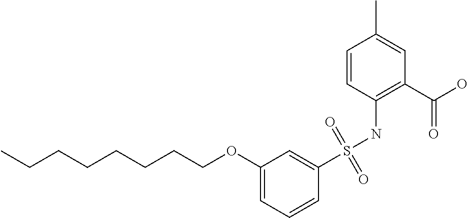 [SMILES: CCCCCCCCOc1cccc(S(=O)(=O)Nc2ccc(C)cc2C(=O)O)c1] |
| 6359 | 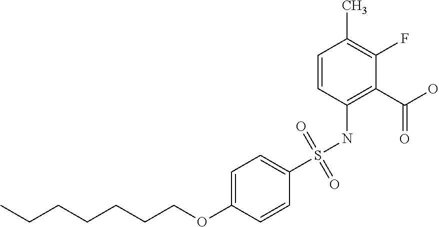 [SMILES: CCCCCCCOc1ccc(S(=O)(=O)Nc2ccc(C)c(F)c2C(=O)O)cc1] |
| 6361 | 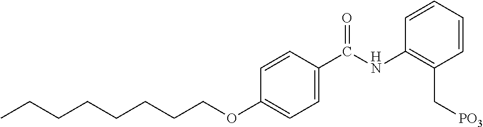 |
| 6363 | 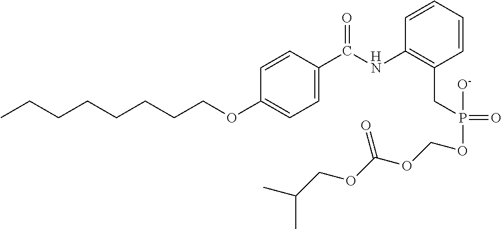 [SMILES: CCCCCCCCOc1ccc(C(=O)Nc2ccccc2CP(=O)([O-])OCOC(=O)OCC(C)C)cc1] |
| 6364 | 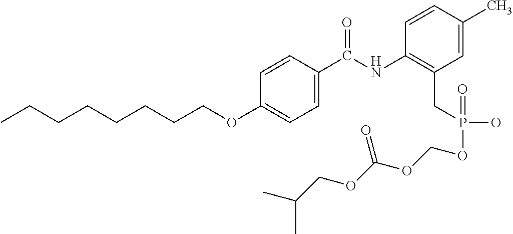 [SMILES: CCCCCCCCOc1ccc(C(=O)Nc2ccc(C)cc2CP(=O)(O)OCOC(=O)OCC(C)C)cc1] |
| 6365 | 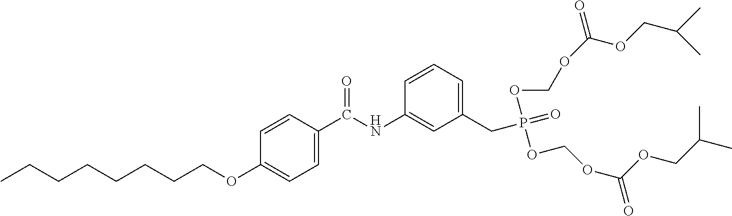 [SMILES: CCCCCCCCOc1ccc(C(=O)Nc2cccc(CP(=O)(OCOC(=O)OCC(C)C)OCOC(=O)OCC(C)C)c2)cc1] |
| 6366 | 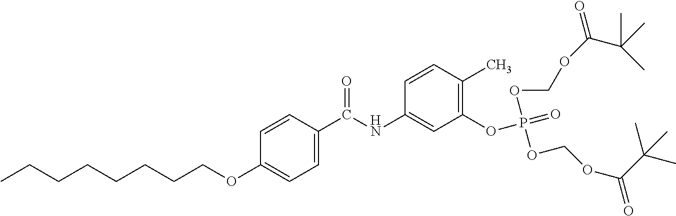 [SMILES: CCCCCCCCOc1ccc(C(=O)Nc2ccc(C)c(OP(=O)(OCOC(=O)C(C)(C)C)OCOC(=O)C(C)(C)C)c2)cc1] |
| 6369 | 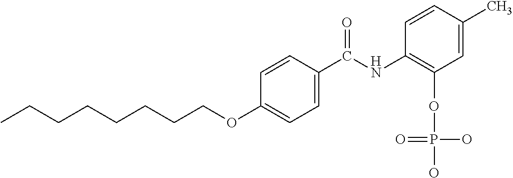 [SMILES: CCCCCCCCOc1ccc(C(=O)Nc2ccc(C)cc2OP(=O)(O)O)cc1] |
| 6421 | 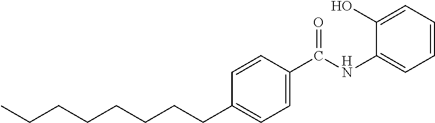 [SMILES: CCCCCCCCc1ccc(C(=O)Nc2ccccc2O)cc1] |
| 6277 | 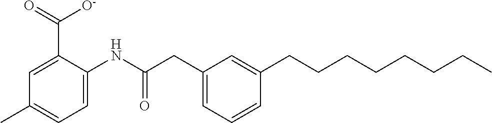 [SMILES: CCCCCCCCc1cccc(CC(=O)Nc2ccc(C)cc2C(=O)[O-])c1] |
| 6329 | 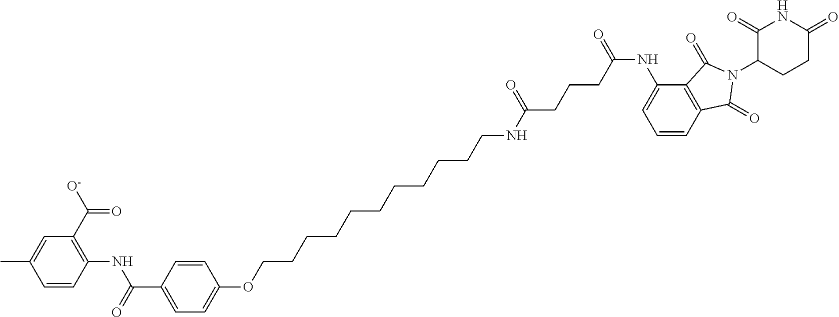 [SMILES: Cc1ccc(NC(=O)c2ccc(OCCCCCCCCCCCNC(=O)CCCC(=O)Nc3cccc4c3C(=O)N(C3CCC(=O)NC3=O)C4=O)cc2)c(C(=O)[O-])c1] |

Disclosed small molecule inhibitors can also encourage apoptosis. For instance, cell cycle analysis and a caspase apoptosis assay have indicated high levels of apoptosis for SCCP ID No. 6359 and SCCP ID No. 6369 in contrast to virtually none for the negative control, suggestive of target engagement for the small molecule inhibitors.

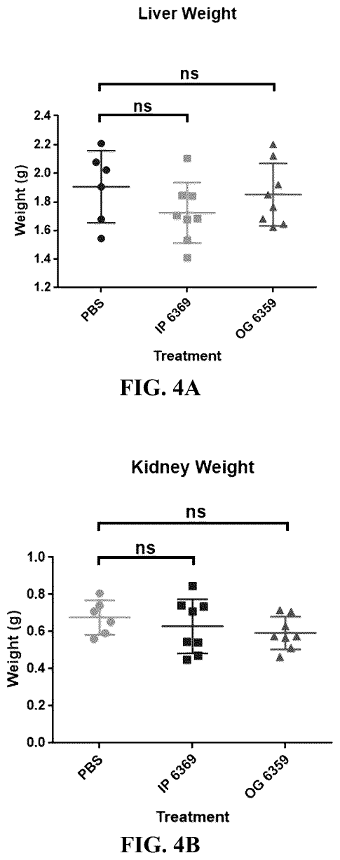

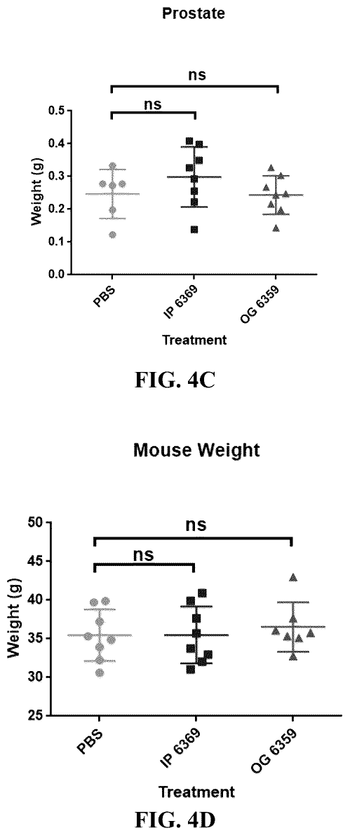

The small molecule inhibitors can be developed and synthesized according to methods as are generally known in the art. For instance, in one embodiment, an iterative combinatorial method can be utilized as described in U.S. Pat. No. 9,175,357 to McInnes, et al., which is incorporated herein by reference. Overall, this combinatorial approach is based upon a known peptide inhibitor and allows both regions of the inhibitor molecule (i.e., the N-cap and the C-cap) to be optimized independently to maximize the affinity and drug-likeness of each component. FIG. 4 provides a general scheme for synthesis according to this particular embodiment. Peptide inhibitors that can be utilized as the basis for development of the small molecule inhibitors can include any peptide inhibitor capable of selectively inhibiting PLK. For instance, the basis peptide inhibitor can include both native peptides and variants thereof.

A benefit of the iterative combinatorial formation approach is the modularity in identification of desired moieties for each subsite. The desired fragments can be combinatorially ligated so that essential compound features will not be compromised during the linking process. The individual fragments can be optimized for potency and drug-like properties and then linked to improve pharmacodynamic and pharmacokinetic properties. This lead optimization stage can be informed by cellular assays and detailed mechanistic studies of anti-tumor effects of the inhibitors. The modularity and combinatorial aspects of this method can allow for the moieties to be exchanged and more narrowly target the characteristics of the physicochemical characteristics, as well as minimize metabolic or toxicophore liabilities, with particularly preferred characteristics depending primarily on the application of the inhibitor (e.g., research, in vivo).

According to one embodiment, following identification and optimization of the end groups, a bridging strategy between the benzamido moiety and an aminobenzoic acid moiety can be selected. For instance, the bridge between the aryl groups can include an amide, sulfonamide, ether, thioether, amine, or carbon-carbon linkages.

The fragments can be subdivided into chemotypes appropriate for docking into the particular PBD sub-sites and prioritized using pharmacophore features to select fragments with desired functionality. High-throughput docking of virtual libraries into the PBD binding site can be performed during the synthesis process. High-throughput docking (HTD) can then be used in refinement of the fragments with calculations including more accurate scoring functions, and interactions filters to limit fragments to those containing the geometrically appropriate functionality. HTD programs such as, without limitation, LIDAEUS, LigandFit, accelrys®, and Glide (Schrödinger®, can incorporate the desired enhancements and can be used to dock virtual fragment libraries into each site. For example, when calculations are parallelized, 100,000 fragments can be screened between 5 and 50 hours depending on the parameterization and number of CPU's employed. Due to inherent inaccuracies with docking, it can be expedient to use different implementations to minimize errors, biases, or incorrect parameters of a single synthesis process. A balance of electrostatic, van der Waals, and H-bonding interactions between each fragment and the binding groove can be used to form the inhibitor having the desired characteristics.

After development of the inhibitor fragments from identified chemotypes, these can be combined in order to substitute all peptidic determinants and form the non-peptidic small molecule inhibitors. Optimization of the fragments can be facilitated using 3-D structures generated through crystallography and flexible molecular docking (using, e.g., Cdocker, accelrys®, etc.) to predict favorable interactions of modified compounds and improve complementarity.

PLK inhibitors disclosed herein may be useful for treating a subject in need thereof. The term “treating” as used herein refers to partially or completely alleviating, improving, relieving, inhibiting progression, and/or reducing incidence of one or more symptoms of a disease, disorder, and/or condition, e.g., cancer.

The term “treating cancer” or “treatment of cancer” may refer to administration of a PLK inhibitor to a subject afflicted with or at risk of a cancerous condition, and may refer to an effect that alleviates the cancerous condition by killing the cancerous cells, but also an effect that result in the inhibition of growth and/or metastasis of the cancer.

In some embodiments, the cancer may include, but is not limited to, adrenal cortical cancer, advanced cancer, anal cancer, aplastic anemia, bile duct cancer, bladder cancer, bone cancer, bone metastasis, brain tumors, brain cancer, breast cancer, childhood cancer, cancer of unknown primary origin, Castleman disease, cervical cancer, colon/rectal cancer, central nervous system (CNS) cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer (NSCLC), small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, and/or Waldenstrom macroglobulinemia. For instance, the cancer may include breast cancer, colon cancer, CNS, leukemia, melanoma, prostate, or renal cancer.

The term “subject” refers to any organism to which aspects of the disclosure can be administered, e.g., for experimental, diagnostic, prophylactic, and/or therapeutic purposes. Subjects to which embodiments of the disclosure can be administered include mammals, such as primates, for example, humans. For veterinary applications, a wide variety of subjects are suitable, e.g., livestock such as cattle, sheep, goats, cows, swine, and the like; poultry such as chickens, ducks, geese, turkeys, and the like; and domesticated animals, such as pets such as dogs and cats. For diagnostic or research applications, a wide variety of mammals are suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

As used herein, the term “administration” refers to introducing a substance (e.g., a PLK inhibitor, abbapolin compound, an exogenous antigen, a cytotoxic agent, etc.) into a subject. The administration thereof can be carried out in any convenient manner, including by aerosol inhalation, injection, ingestion, transfusion, implantation, or transplantation. For instance, the cannabinoid compound may be administered orally, subcutaneously, intravenously, or intratumoral. In this regard, “oral” administration can refer to administration into a subject's mouth; “subcutaneous” administration can refer to administration just below the skin; “intravenous” administration can refer to administration into a vein of a subject; and “intratumoral” administration can refer to administration within a tumor.

Pharmaceutical compositions disclosed herein may be formulated to be compatible with its intended route of administration. As used herein, “routes of administration” may include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition can be sterile and should be fluid to the extent that easy syringability exists. It can be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. Prolonged absorption of the injectable compositions may be brought about by including in the composition an agent which delays absorption, for example, aluminum monostearate and gelatin.

Oral compositions may include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Compositions for parenteral delivery, e.g., via injection, can include pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., corn oil) and injectable organic esters such as ethyl oleate. In addition, the composition can contain minor amounts of auxiliary substances such as wetting or emulsifying agents, pH buffering agents and the like that can enhance the effectiveness of the phenolic compound. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents, and dispersing agents.

In one embodiment, a therapeutically effective amount of the PLK inhibitor may be administered to the subject. The term “therapeutically effective amount” refers to those amounts that, when administered to a subject in view of the nature and severity of that subject's disease or condition, will have a desired therapeutic effect, e.g., an amount which will cure, prevent, inhibit, or at least partially arrest or partially prevent a target disease or condition. A therapeutically effective dose further can refer to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

A therapeutically effective dose can depend upon a number of factors known to those of ordinary skill in the art. The dosage can vary depending upon known factors such as the pharmacodynamic characteristics of the active ingredient and its mode and route of administration; time of administration of active ingredient; identity, size, condition, age, sex, health and weight of the subject or sample being treated; nature and extent of symptoms; kind of concurrent treatment, frequency of treatment and the effect desired; and rate of excretion. These amounts can be readily determined by the skilled artisan.

The therapeutically effective amount is at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight.

In some embodiments, for instance, the PLK inhibitor (e.g., abbapolin) may be administered to a subject at a dosage of about 20 mg/kg body weight to about 200 mg/kg body weight, such as from about 35 mg/kg body weight to about 175 mg/kg body weight, such as from about 50 mg/kg body weight to about 150 mg/kg body weight, such as from about 75 mg/kg body weight to about 125 mg/kg body weight, or any range therebetween.

Pharmaceutical compositions as described herein can be administered to the subject one time (e.g., as a single injection or deposition). Alternatively, administration can be once or twice daily to a subject in need thereof for a period of from about 2 days to about 35 days, such as from about 7 days to about 28 days, such as from about 10 to about 21 days, or any range therebetween. It can also be administered once or twice daily to a subject for a period of 1, 2, 3, 4, 5, 6, 7, 8, 9, 10, 11, 12 times per year, or a combination thereof.

The present disclosure may be better understood with reference to the Examples, set forth below.

### EXAMPLE 1

### Materials and Methods

### Fluorescence Polarization Assay

Materials were dissolved in DMSO (10 mM) and diluted from 10 nM to 600 μM (maximum of 6% DMSO tolerance in the assay). PLK1 PBD (367-603) and PLK3 PBD (335-646) proteins were obtained from BPS Biosciences Inc. (San Diego, CA); 17 ng PLK1 and 156 ng PLK3 were used per reaction. The fluorescein-tracer phospho-peptides (MAGPMQS[pT]PLNGAKK (SEQ ID NO: 1) for PLK1, and GPLATS[pT]PKNG (SEQ ID NO: 2) for PLK3) were used at a final concentration of 10 nM. Incubation was carried out at room temperature for 45 minutes. Fluorescence was measured using a DTX 880 plate reader and Multimode Analysis software (Beckman Coulter, Brea, CA). The polarization values in millipolarization (mP) units were measured at an excitation wavelength of 488 nm and an emission wavelength of 535 nm. Each data point was performed in triplicate for every experiment, and experiments were performed at least three times. An IC50 value for each compound was calculated from linear regression analysis of the plots.

### PLK1 Kinase Inhibition Assay

The CycLex® Polo-like Kinase 1 Assay/Inhibitor Screening Kit was used to measure catalytic inhibition (MBL Life Science, Nagano, Japan). This ELISA assay measures the catalytic activity of full length PLK1 for a defined substrate, which is detected by an anti-phospho-threonine antibody (PPT-07) and peroxidase coupled secondary antibody. Plates pre-coated with a Threonine-containing substrate were incubated with PLK1, kinase buffer containing 7.5 μM ATP (modification from recommended concentration), in the absence or presence of increasing concentration of inhibitor. After incubation, the phosphorylated substrate resulting from PLK1 kinase activity was detected using the PPT-07 antibody and horseradish peroxidase conjugated anti-rabbit IgG antibody. Peroxidase catalyzes the conversion of the colorless solution to yellow, which was quantified using a DTX 880 plate reader. Absorbance measurements (450 nm) were plotted to calculate activity in each sample, and % inhibition was calculated in the wells relative to activity in the absence of inhibitor.

### Cell Culture

HeLa cervical cancer cells were obtained from ATCC (Manassas, VA). Histone H2B GFP-labeled HeLa cells (HeLa-H2B-GFP) were kindly provided by Dr. Geoffrey Wahl (Gene Expression Laboratory, Salk Institute), and were confirmed as >95% GFP positive by FACS (data not shown) but were not otherwise authenticated. Cells were maintained in DMEM (Invitrogen™, Carlsebad, CA) supplemented with 10% FBS or Corning® Nu-serum™ (BD™ Biosciences, Franklin Lakes, NJ) and 1% penicillin/streptomycin (Invitrogen™) in a humidified incubator and 5% CO₂ at 37° C.

PC3 prostate cancer cells were maintained in DMEM (Invitrogen™, Carlsebad, CA) supplemented with 10% FBS or Corning® Nu-serum™(BD™ Biosciences, Franklin Lakes, NJ) and 1% penicillin/streptomycin (Invitrogen™) in a humidified incubator and 5% CO₂ at 37° C.

A-549 lung cancer cells were maintained in Hams F-12 1× with glutamine (Corning® Cellgro, Manassas, VA), and supplemented with 10% FBS or Corning® Nu-serum™ (BD™ Biosciences, Franklin Lakes, NJ) and 1% penicillin/streptomycin (Invitrogen™) in a humidified incubator and 5% CO₂ at 37° C.

HCT-116 (p53 +1+and −/−) colon cancer cells were maintained DMEM (Invitrogen™ Carlsebad, CA) supplemented with 10% FBS or Nu-serum (BD™ Biosciences, Franklin Lakes, NJ) and 1% penicillin/streptomycin (Invitrogen™) in a humidified incubator and 5% CO₂ at 37° C.

Retinal Pigment Epithelial (RPE) cells were maintained in 1:1 DMEM/Hams F-12 (Invitrogen™, Carlsebad, CA; Corning® Cellgro, Manassas, VA), and supplemented with 10% FBS or Corning® Nu-serum™ (BD™ Biosciences, Franklin Lakes, NJ) and 1% penicillin/streptomycin (Invitrogen™) in a humidified incubator and 5% CO₂ at 37° C.

### Cell Viability Assay

Exponentially growing cells were plated in 96-well dishes. Dose response curves were used to treat cells for 72 hours with inhibitors. Following the three-day treatment, cell viability was measured using the MTT (3-(4,5-dimethylthiazol-2-yl)-2,5-diphenyltetrazolium bromide) colorimetric assay. Cells were incubated for 3 hours with MTT allowing viable cells to metabolize the tetrazolium dye to a purple colored solution. Absorbance measurements (595 nm) were quantified using a DTX 880 plate reader and IC₅₀ values were generated for FLIPs.

### Analysis of Cell Cycle Progression by FACS

### Serum-Starved Synchronization

Cells were synchronized in G1 by serum starvation. Briefly, exponentially growing cells were plated overnight. Media (DMEM, 10% FBS, 1% Pen. Strep.) was then removed and replaced with media containing no FBS. Cells were starved for 72 hours then treated for 24 hours with inhibitor. Following treatment, cells were harvested and processed for endpoint experimental analysis.

### BrdU Labeling/FACS

Ethanol fixative was removed by centrifugation and the cells were washed with 1 mL ice-cold PBS/1% BSA. Cells were denatured by resuspending in 0.2 mg/mL pepsin in 2N HCl and incubated in 37° C. water bath for 15 minutes. Hydrolysis was then terminated by adding 1 M Invitrogen™ Novex° Tris-Glycine. Cells were then washed with PBS/1% BSA then the primary anti-BrdU antibody (1:100 dilution in TBFP; 0.5% Tween-20, 1% BSA, 1% FBS, PBS) was added and cells were incubated for 25 minutes in the dark, at room temperature. Cells were subsequently washed with PBS/1% BSA then allowed to incubate for 25 minutes in the secondary fluorescent antibody (1:200 dilution Alexa Fluor® 488 F(ab′)₂ fragment of goat anti-mouse IgG). Cells were washed once more with PBS/1% BSA and re-suspended in DAPI for 30 minutes before being analyzed by flow cytometry.

### Cellular Thermal Shift Assay (CETSA)

Cellular thermal shift assay (CETSA) was performed with PC3 cells cultured in RPMI medium supplemented with 10% FBS. For an initial determination of the melting profile of PLK1 AND PLK3, cells dispensed into 96-well PCR plates in the above medium (6000 cells/well in 50 μL) were subjected to temperature gradient (38-60° C.) for 10 min. Cold non-denaturing lysis buffer (PBS supplemented with 0.1% TritonX-100 and 1×protease inhibitors) was added to wells, and the plate was rocked, then incubated for 15 min on ice. Centrifugation was performed at 14000 rpm to sediment the denatured protein content. Supernatant was collected and subjected to SDS-PAGE, and immunodetection was performed using anti-PLK1 and anti-PLK3 antibodies. PLK1 and PLK3 bands were quantified on a LI-COR C-Digit Blot Scanner, and Tagg and Tagg values were calculated for both proteins. In subsequent experiments, cells were treated at increasing doses (3.0, 1.0, 0.3, 0.1, 0.03 μM) of inhibitors together with DMSO control, for 2 h. Cells were then subjected to heat shock at Tagg for 10 min, and unstable protein was removed by centrifugation step. Following immuno-blotting, bands of stable PLK1 and PLK3 were quantified, normalized to loading control and plotted using GraphPad Prism software. EC50 values for inhibitors were calculated. In an initial heat gradient, PLK1 displayed a temperature-dependent decay with a Tagg of 56.2° C., whereas PLK3 displayed a temperature-dependent decay with a Tagg at 48.0° C. Subsequently, dose-dependent potency of each inhibitor was tested at Tagg, the temperature point at which 50% of the target protein is denatured. The EC50 of the peptides were determined from the curve fit of the dose response.

### EXAMPLE 2

### Abbapolins Inhibit PLK and Induce its Degradation

Drug like compounds were discovered utilizing the REplacement with Partial Ligand Alternatives through Computational Enrichment (REPLACE) strategy. REPLACE is a strategy involving the search for “fragment alternatives” for peptide determinants in an iterative fashion. It involves 1) truncation of peptide determinants, 2) docking of small molecule fragments into the empty sub-pocket in the crystal structure to prioritize them and 3) synthesis of small molecule-peptide hybrid compounds. Utilizing this structure-guided approach, abbapolins were discovered that bind tightly to PLK1 through the polo-box domain (PBD) and are able to block PLK1 specific functions. 4-alkybenzamide derivatives, abbapolins ((2-(4-AlkylBenzamido) Benzoic Acids)), induce proteasome mediated degradation of the PLK1 protein in a dose dependent manner without the addition of a E3 ligase recruiting ligand to promote ubiquitination. This allows for PROTAC like pharmacology without the inherent complexity and lack of drug-likeness of this class of compounds. Degradation will eliminate all functions of PLK1 potentially overcoming lack of clinical efficacy observed with only blocking catalytic activity and potentially be more difficult to develop resistance to.

Abbapolins interact with the cryptic hydrophobic pocket of the PBD in a way that is incompatible with formation of dimeric PLK1 observed to occur through the L505. Cryptic binding sites are absent in unbound proteins but present in ligand-bound structures. Mutational studies of residues in the PBD suggest that some substrates bind differently to the PBD and in this way PLK1 activity can be directed to specific mitotic structures. Cryptic pocket mutations selectively impair PLK1 binding to the kinetochore phosphoprotein PBIP1 but not to the centrosomal substrate NEDD1. Furthermore, the CP as revealed in the phospho-PON dimer structure plays a key role in PLK1 dimerization. L505 interacts with this pocket adjacent to the phosphopeptide binding motif and plays a key role in stabilization of PLK1 dimers. Modeling studies disclosed herein indicate that the dimer is incompatible with binding of the abbapolins as the hydrophobic tail interacts with the CP and therefore should preclude dimer formation involving L505 (FIG. 1). Without wishing to be bound by theory, it is likely that abbapolins disrupt dimeric PLK1 and further that their ability to do this is related to their mechanism of inhibition including the observed effects on PLK1 stability.

### EXAMPLE 3

### PBD-Inhibitors Bind Potently to the PLK1 -PBD and are Selective

Substructure searching for 4-alkybenzamide derivatives in commercial libraries and subsequent testing identified one lead molecule inhibitor, SCCP ID No: 5881. From this lead, the structure activity relationship (SAR) of more than forty small molecules was analyzed. The total of these compounds can be found in FIG. 1. Specifically, compound binding to PLK1 (discussed first) and PLK3 (discussed second) in vitro was analyzed. ATP inhibition was also measured and compound activity analyzed in cancer cells.

### Structure Activity Relationship of C-Capping Group on Benzamide Small Molecules

A 4-alkyl substituted benzoic acid fragment was used as a template for the identification of compounds containing alternatives for the C-terminal residues (‘C-caps’) and a library of commercially available derivatives was screened computationally and in FP binding assay. Extending the alkyl tail from butyl (C4) to decyl (C10) improved the affinity for the PLK1 PBD from >600 μM down to 21 μM (Table 1, 6153-6287). To explore synthetic versatility and improve solubility, heteroatoms linking the alkyl tail in binding to PLK PBDs were incorporated and the PBD binding order of S>C>O>N. Further improvement in binding and cellular activity were obtained through substitution of the Ccap (6165), through addition of a phosphate isostere and through use of a sulfoamide linking group. These compounds possess MTT values in PC3 PC cells from >120 μM (6153) down to 9.2 μM (6184) and correlated well with PBD binding (Table 2).

TABLE 2

| | | | PLK1 | PLK3 | |
|---|---|---|---|---|---|
| SCCP | | | PBD | PBD | PC3 IC50 |
| ID | R1 | R2 | IC50 (μM) | IC50 (μM) | (μM) |
| 6153 | H | N | >600 | >600 | >120 |
| 6154 | H | N | >600 | | >120 |
| 6165 | H | C₈ | 23.5 ± 4.9 | 37.6 ± 5.7 | 14.2 ± 2 |
| 6171 | H | C₈N(Me) | >600 | | 27.3 |
| 6172 | H | C₈N(Me) | >600 | | 27.96 |
| 6184 | H | C₈ | 95.29 | | 9.2 ± 2 |
| 6189 | H | C₆CONH | 37% inhi. at | | |
| | | | 400 μM | | |
| 6195 | H | C₈ | 51.52 | | 20.4 ± 4.4 |
| 6237 | H | C₉H₉ClNOS | | | |
| 6277 | C₈ | H | 19.7 | 29.4 | |
| 6279 | OH | C₈ | 31.8 | | |
| 6280 | OCH3 | C₈ | >100 | >200 | 25.9, 18.3 |
| 6281 | C₈ | H | 19.49 | | |
| 6282 | CH3 | C₈ | 21 | | |
| 6286 | H | C₈ | 52.98 | | 26.6, 25.1 |
| 6287 | H | C₈ | 80.76 | | 50% inhi. at |
| | | | | | 100 μM |

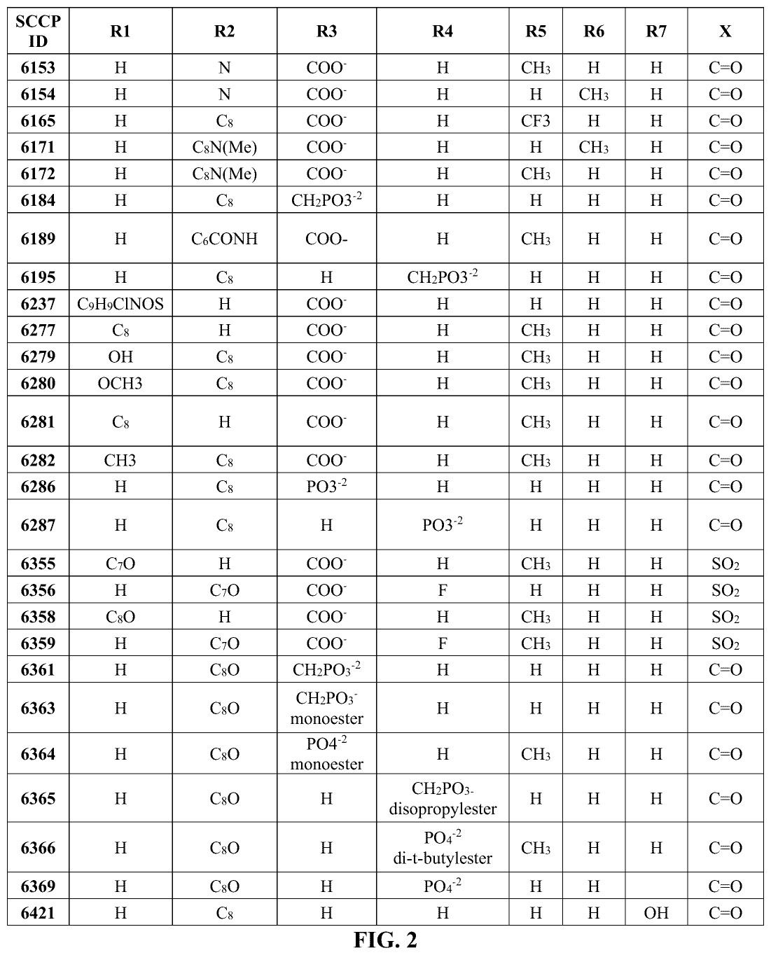

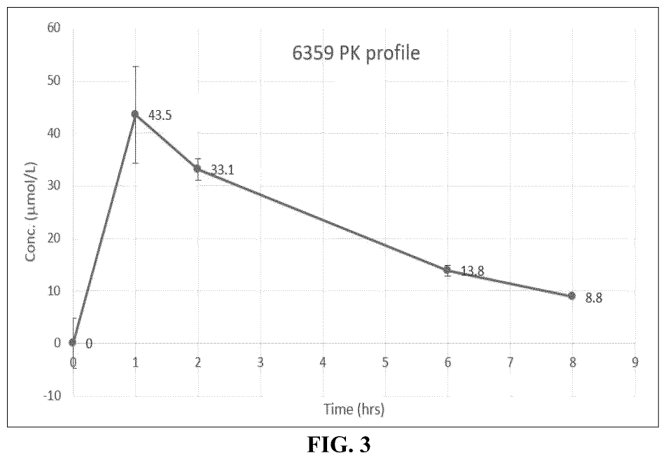

To further optimize affinity of the abbapolins a number of phosphate isosteres were incorporated to determine if activity can be enhanced through more optimal interactions with residues forming ion pairing interactions with the pThr of the peptide. In modeled structures, the ortho carboxylate interacts with Lys540 but not with His538. Both of these residues interact with the phosphopeptide and therefore additional contacts to His538 would improve affinity. The abbapolin analog 3-(4-octylbenzamido)phenyl phosphate analog (6369) was synthesized and shown to have much improved cellular activity with an IC₅₀ of 2.7 μM in PC3 cells demonstrating that this group has a profound effect on binding to the PBD. Further analogs can include tetrazole, sulfonamide, sulfonate, and phosphonate, all of which are acidic and can interact with positively charged residues. The geometry and relative orientation of the N and C capping groups were optimized through linking functional groups incorporating amide replacements, e.g. sulfonamide, amine (FIG. 2). Abbapolin 6359 incorporates a sulfonamide group linking an octyloxybenzoic acid Ncap and a 2-amino-6-fluorobenzoic acid Ccap. This was shown to be the most potent non-phospho containing compound with an antiproliferative activity of 11.3 μM in PC3 cells. The 6-fluoro substituent increases the acidity of the benzoic acid promoting electrostatic interactions with the phosphate interacting residues.

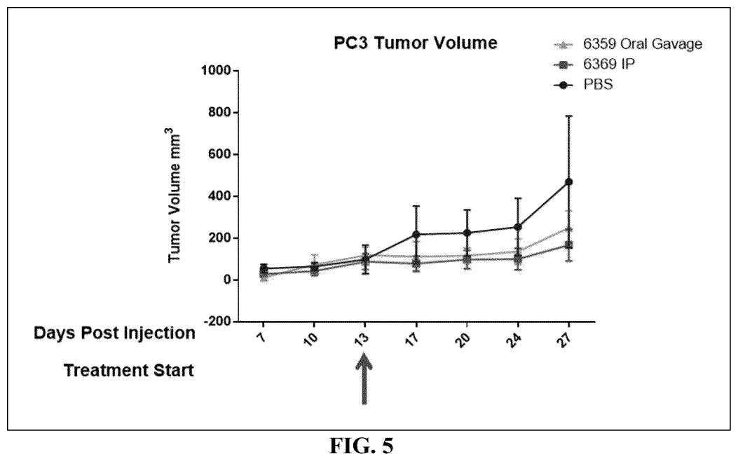

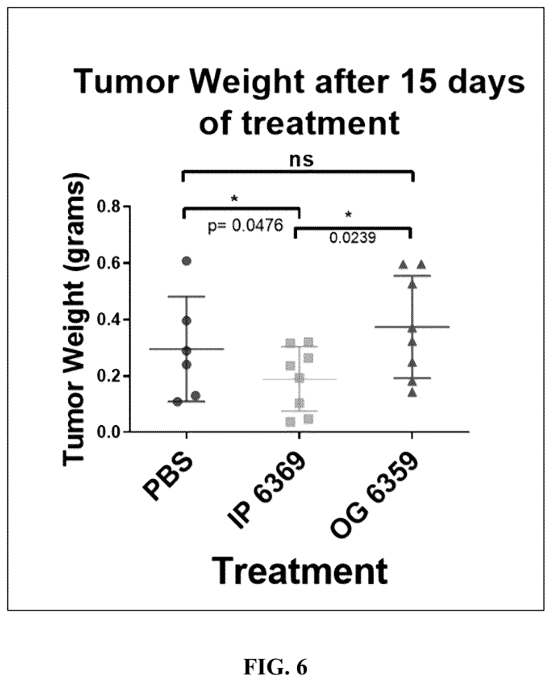

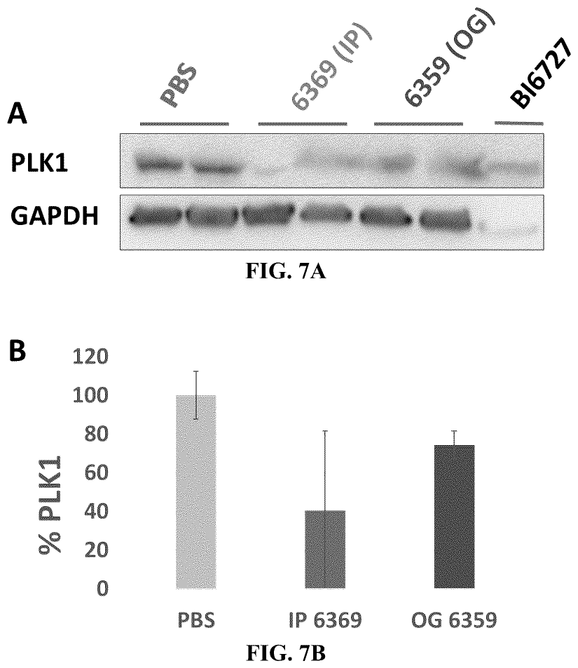

The efficacy of promising abbapolin compounds were tested in multiple cancer cell lines. An in vivo mouse xenograft study showed that abbapolins were well tolerated by mice as no sign of toxicity was observed and resulted in no loss of body weight, nor was there a change in liver, kidney, and prostate weight observed (FIGS. 4A-D) (P>0.05, non-significant). To do so, athymic nude mice were injected in their flanks with 2.7×10⁶ PC3 prostate cancer cells. After the tumors established at 13 days, 5-8 mice per group were treated with 6359 and 6369 daily for 5 days (M-F) for a total of 15 days. Due to its good PK values, 6359 was administered by oral gavage at 100 mg/kg. Because of its low oral absorption, 6369 was administered IP at the limit of its solubility in PBS, which was 40 mg/kg. Both abbapolins showed ability to slow the growth of PC3 tumors over the 3-week period at the doses specified, which demonstrates in vivo efficacy of abbapolins in prostate cancer cells. The phospho analog (6369; EC50 PLK1=0.15 μM, PC3 IC50=2.7 μM) and the sulfonamide abbapolin derivative (6359, 11 μM) inhibited the prostate xenograft tumors growth but only 6369 showed statistically significance (p<0.0476) compared to PBS. This is in agreement with the PLK1 degradation in the xenograft tumor. 6369 caused significantly more PLK1 loss compared to PBS and 6359 (FIG. 5). This indicates that inhibition of the PC3 tumor growth was because of PLK1 degradation caused by abbapolins.

Both compounds have measurable blood levels when dosed at 50 mg/kg by oral gavage (6369, 6359 FIG. 5). 6359 (carboxylate isostere of phosphate) had about 1000-fold higher oral absorption compared to the phosphate analogue and reached blood levels almost 4 times its cellular IC50 (11 μM). 6359 Cₘₐₓ was 43.5 μM, the AUC was 22 μmol/hr/L and the t_{1/2} was 3.1 hours. These reasonable PK values of the lead abbapolin compounds tested in vivo are promising and indicate optimization will lead to further improvements.

Lead abbapolin compounds were tested in multiple cancer cell lines, including non-small cell lung cancer (NSCLC) cells, melanoma cells, colon cancer cells, central nervous system (CNS) cancer cells, prostate cancer cells, renal cancer cells, and breast cancer cells. Each of these cancer cells lines were sensitive to the lead abbapolin compounds in a cell viability assay, including 6184 (Table 3), 6277 (Table 4), 6279 (Table 5), 6355 (Table 6), 6359 (Table 7), 6369 (Table 8), and 6421 (Table 9). Surprisingly, after 48 hours of treating with 6277, only 3.4% of cells remained in the LOX IMVI cell line indicating 96.4% inhibition of PLK1 (Table 4). Similarly, only 8.57% of NSCLC cells remained in the HOP-92 cell line after treatment with 6359 indicating 91.43% inhibition.

TABLE 3
6184 (single dose 10 μM)

| Cancer Type | Cancer Cell Line | Cells Remaining (%) |
|---|---|---|
| NSCLC | HOP-92 | 43.86 |

TABLE 4
6277 (single dose 25 μM)

| Cancer Type | Cancer Cell Line | Cells Remaining (%) |
|---|---|---|
| Melanoma | LOX IMVI | 3.4 |
| Colon | HCT-15 | 13.2 |
| Leukemia | MOLT-4 | 17.93 |
| NSCLC | NCI-H460 | 20.45 |
| Leukemia | RPMI-8226 | 25.43 |
| Leukemia | CCRF-CEM | 26.74 |
| Colon | HCT-116 | 28.14 |
| Leukemia | K-562 | 28.94 |
| Leukemia | SR | 31.08 |
| NSCLC | A549/ATCC | 31.38 |

TABLE 5
6279 (single dose 25 μM)

| Cancer Type | Cancer Cell Line | Cells Remaining (%) |
|---|---|---|
| CNS | SNB-19 | 43.49 |
| CNS | U251 | 45.14 |
| Leukemia | K-562 | 49.7 |
| Colon | KM12 | 52.19 |
| Leukemia | CCRF-CEM | 55.59 |
| Colon | HCT-15 | 56.83 |
| NSCLC | HOP-92 | 57.85 |
| Prostate | PC-3 | 58.63 |
| CNS | SF-295 | 59.38 |
| NSCLC | NCI-H522 | 59.89 |
| Renal | ACHN | 61.79 |
| Leukemia | RPMI-8226 | 63.91 |

TABLE 6
6355 (single dose 25 μM)

| Cancer Type | Cancer Cell Line | Cells Remaining (%) |
|---|---|---|
| Melanoma | SK-MEL-2 | 30.01 |
| Colon | HCT-15 | 49.94 |
| Leukemia | RPMI-8226 | 51.41 |
| Breast | MDA-MD-468 | 55.12 |
| NSCLC | HOP-92 | 57.02 |
| CNS | SF-295 | 59.2 |
| Melanoma | SK-MEL-5 | 59.55 |
| Melanoma | UACC-62 | 61.59 |
| CNS | U251 | 64.99 |
| NSCLC | NCI-H522 | 65.09 |
| Leukemia | CCRF-CEM | 67.09 |

TABLE 7
6359 (single dose 10 μM)

| Cancer Type | Cancer Cell Line | Cells Remaining (%) |
|---|---|---|
| NSCLC | HOP-92 | 8.57 |
| Leukemia | RPMI-8226 | 54.91 |

TABLE 8
6369 (single dose 10 μM)

| Cancer Type | Cancer Cell Line | Cells Remaining (%) |
|---|---|---|
| Leukemia | CCRF-CEM | 24.97 |
| NSCLC | NCI-H522 | 33.06 |
| Leukemia | RPMI-8226 | 39.13 |
| NSCLC | HOP-92 | 43.19 |
| Renal | UO-31 | 46.64 |
| Leukemia | HL-60(TB) | 54.82 |
| Leukemia | MOLT-4 | 58.19 |

TABLE 9
6421 (single dose 25 μM)

| Cancer Type | Cancer Cell Line | Cells Remaining (%) |
|---|---|---|
| Breast | T-47D | 27.32 |
| Breast | MCF7 | 35.21 |
| Leukemia | CCRF-CEM | 37.43 |
| Melanoma | SK-MEL-2 | 38.46 |
| Breast | MDA-MB-468 | 45.03 |
| Leukemia | RPMI-8226 | 48.98 |
| NSCLC | EKVX | 52.89 |
| Leukemia | K-562 | 53.71 |
| Leukemia | MOLT-4 | 58.2 |
| Renal | UO-31 | 59.4 |
| NSCLC | NCI-H23 | 62.07 |
| NSCLC | NCI-H226 | 66.21 |
| Leukemia | HL-60(TB) | 69.66 |
| Melanoma | UACC-62 | 69.99 |
| Melanoma | SK-MEL-5 | 70.42 |
| NSCLC | NCI-H522 | 70.52 |

### EXAMPLE 4

In vitro binding affinity of abbapolins 6369 and 6359 was measured in HeLa cervical cancer cells and PTEN deficient prostate cancer (PC3). A synthetic lethal interaction has been demonstrated between PLK1- and PTEN-deficient prostate cancer cells. A differential sensitivity was also observed depending on PTEN status. Specifically, PTEN null PC3 colon cancer cells were more sensitive to the abbapolin compounds (Table 10).

TABLE 10

| | Compound ID | | p53 | PTEN |
|---|---|---|---|---|
| | 6369 | 6359 | status | status |
| PC3 IC₅₀ (μM) | 2.3 ± 0.9 | 11.3 ± 1.8 | MT | null |
| HeLa IC₅₀ (μM) | 17.5 ± 2.9 | 18.2 | MT | WT |
| in vitro binding IC₅₀ (μM) | 115.2 | 65.5 | | |

### EXAMPLE 5

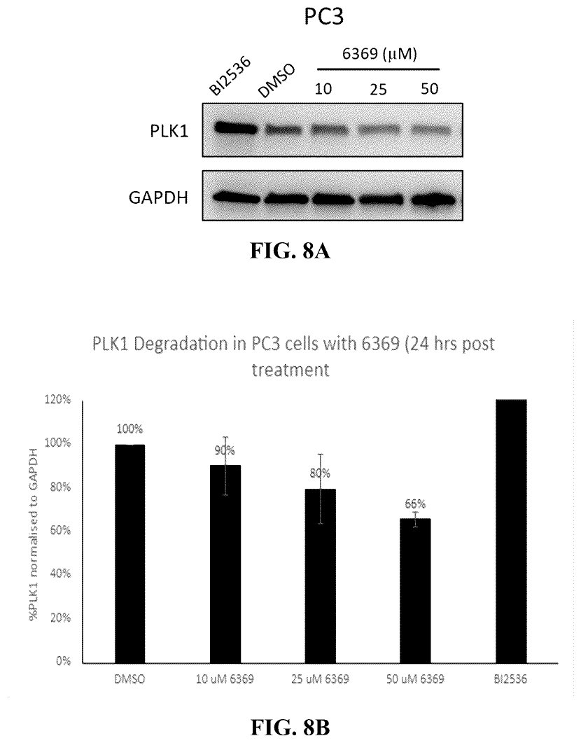

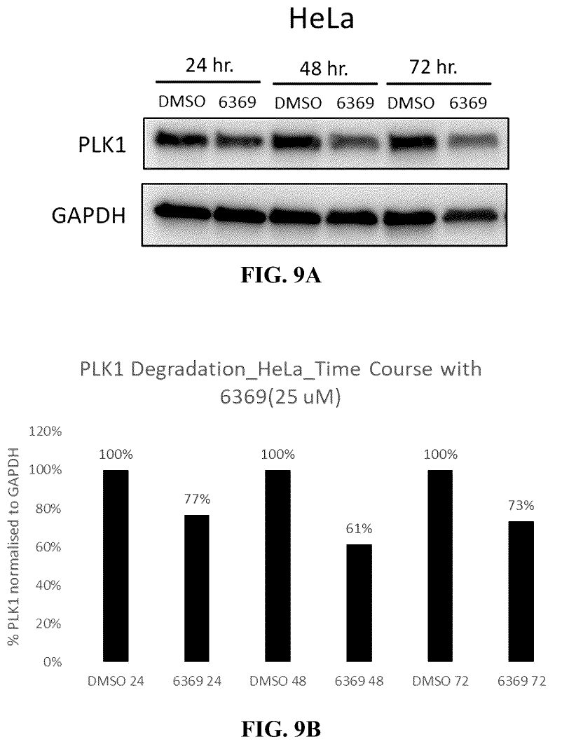

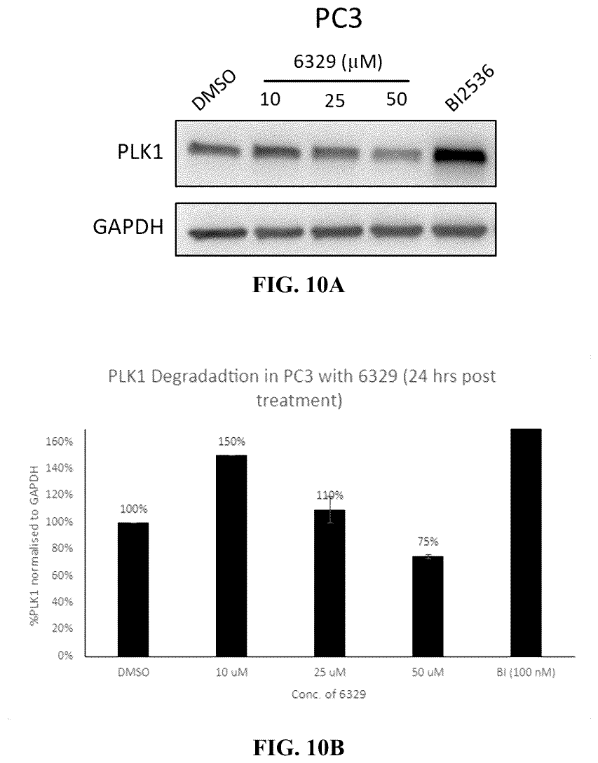

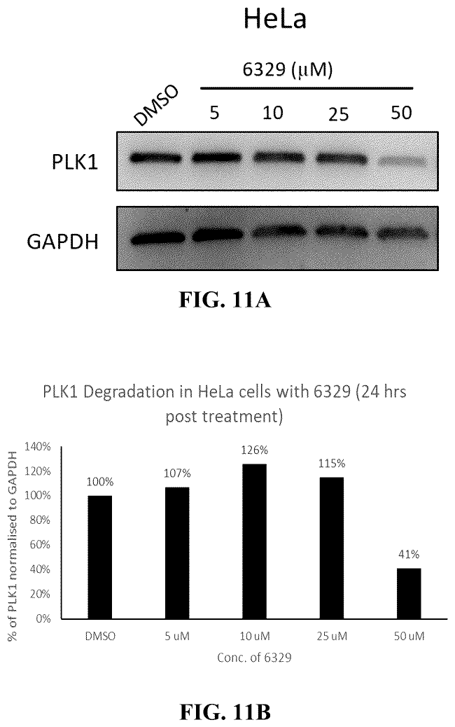

PLK1 degradation by abbapolin compounds was observed in PC3 and HeLa cells. For example, PC3 cells were treated with varying dosages of abbapolin 6369, and percent of PLK1 degradation was measured at 24 hours (FIGS. 8A-8B). HeLa cells were treated with 25 μM abbapolin 6369, and time points shown in the assay were 24 hours, 48 hours, and 72 hours (FIGS. 9A-9B). PC3 cells were treated with varying dosages of abbapolin 6329, and percent of PLK1 degradation was measured at 24 hours (FIGS. 10A-11B). HeLa cells were treated with varying dosages of abbapolin 6329, and time points shown in the assay were 24 hours, 48 hours, and 72 hours (FIGS. 11A-11B).

### EXAMPLE 6

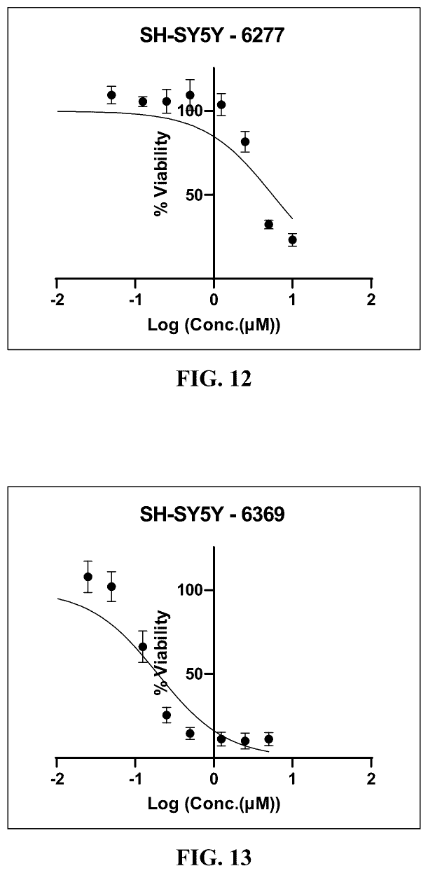

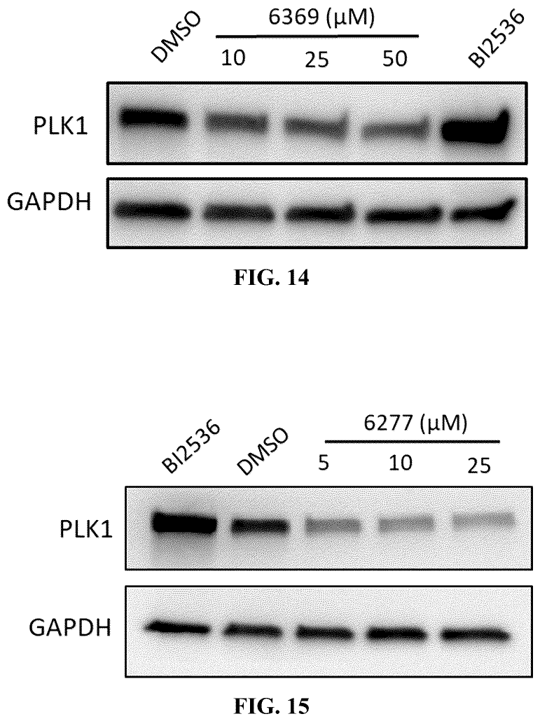

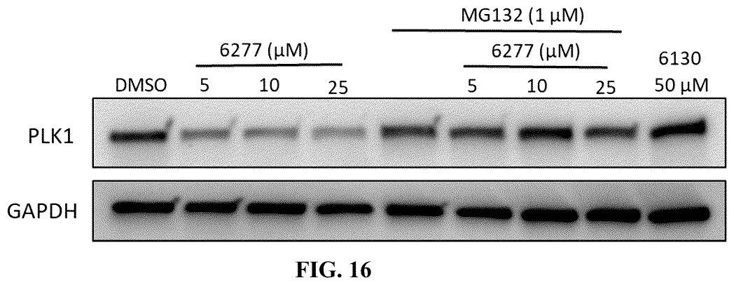

Abbapolin compounds activity was evaluated in neuroblastoma cells. Neuroblastoma cells (SH-SY5Y) overexpressing PLK1 were sensitive to treatment of abbapolin 6277 (IC₅₀=5.25±0.5 μM; FIGS. 12) and 6369 (232±55 μM; FIG. 13). Further, abbapolins induced PLK1 degradation in SH-SY5Y cells (FIG. 14-16).

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure, which is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present disclosure.

What is claimed is:

1. A method for treating a cancer, the method comprising administering to a subject in need thereof a therapeutically effective amount of a compound of Formula (I): 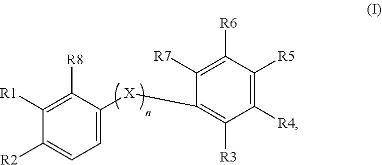
or a pharmaceutically acceptable salt or stereoisomer thereof, wherein:
R1 comprises a hydrogen, —C1 to C8 alkyl, —C1 to C8 alkoxy, or —OH;
R2 is independently selected from hydrogen, (C1 to C8) alkyl, (C1 to C8) alkoxy, secondary (C1 to C8) alkyl methylamine (C8N(Me)), —CONH—(C1 to C6 alkyl), (C1 to C9 alkyl)-sulfinylamino chloride, or NH₂;
R3 through R8 are independently selected from carboxyl, hydrogen, alkyl phosphonate, phosphonate, alkyl phosphonate monoester, phosphate, halogen, alkyl phosphonate disopropylester, phosphate di-t-butylester, —SO₂F, —OSO₂F, —NSO₂F, di-tert-butyl N,N-diethylphosphoramidite, methyl pivalate, or isobutyl methyl carbonates;
X is carbonyl, sulfonyl, amide, —C1 to C4 alkyl-NH—, —NH—, —C1 to C4 alkyl-S, —SONH—, or —SO₂N—;
n is 0, 1, or 2.

2. The method of claim 1, wherein the compound or its pharmaceutically acceptable salt is a PLK inhibitor.

3. The method of claim 1, wherein the compound or its pharmaceutically acceptable salt has the structure of one or more of:

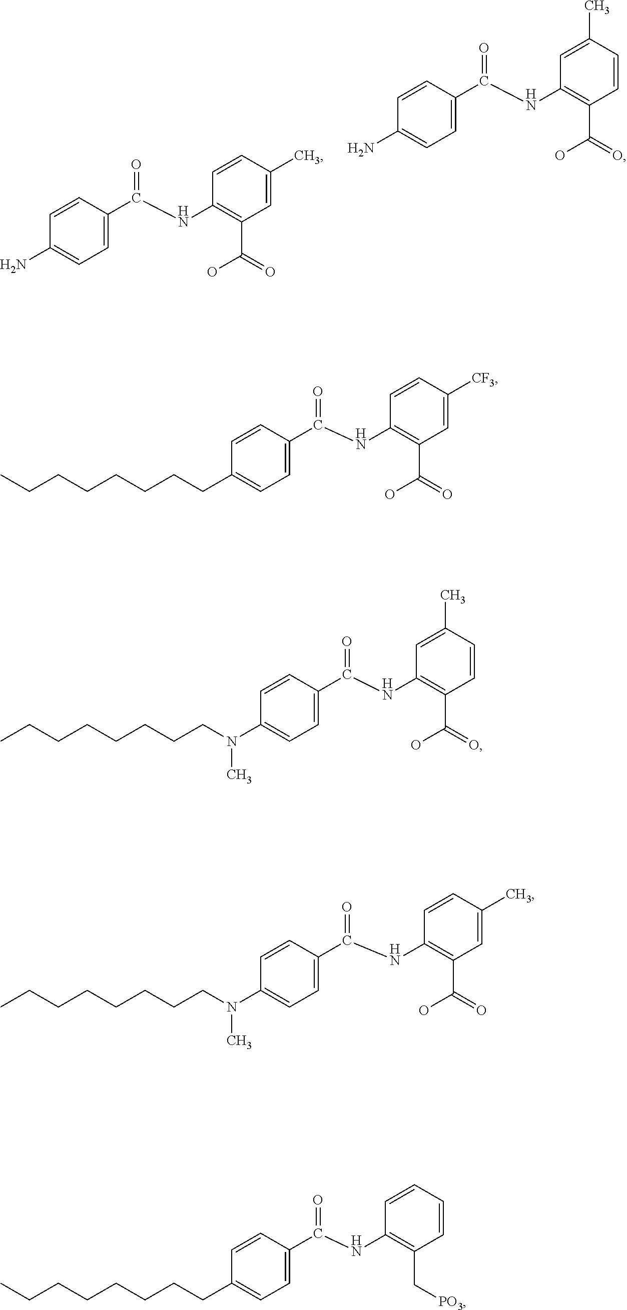

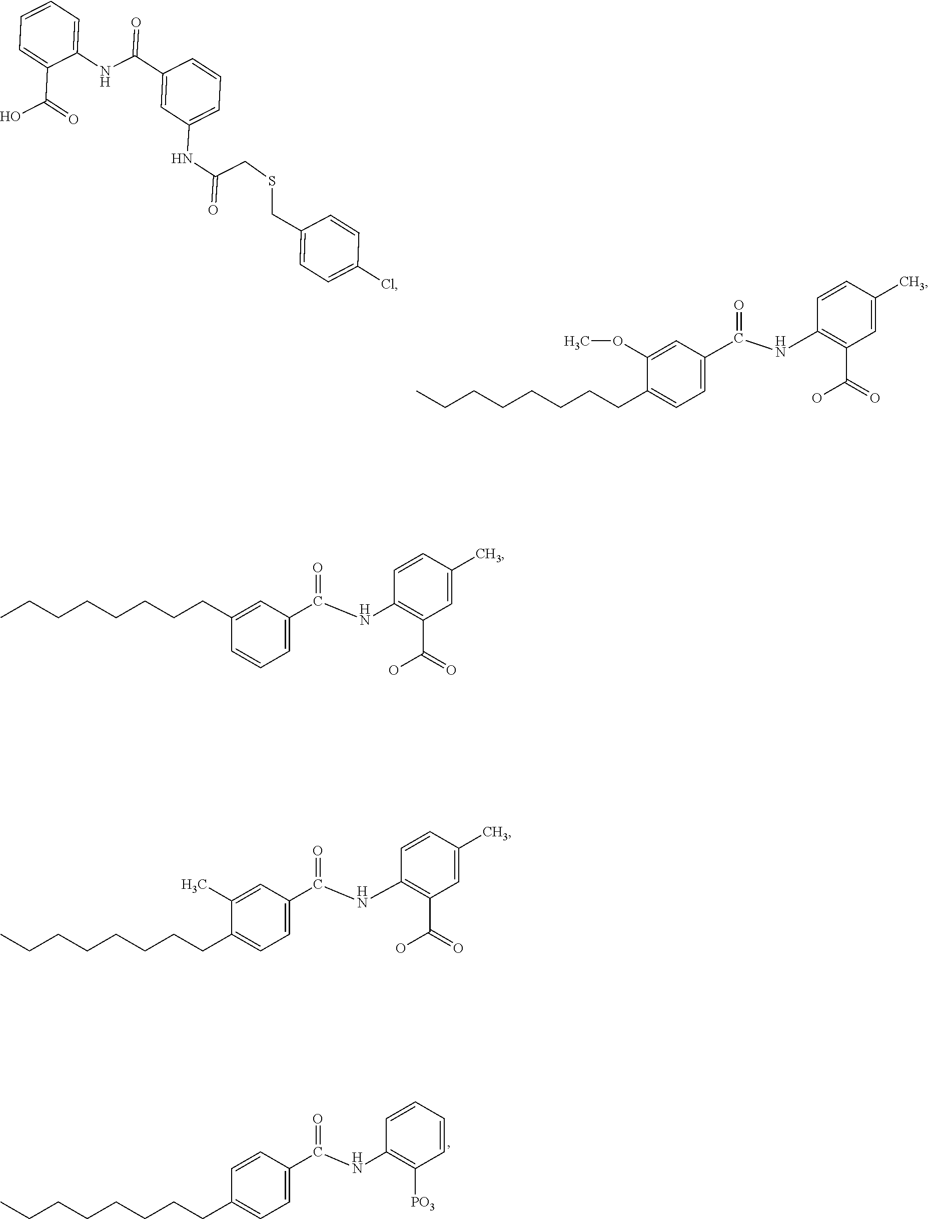

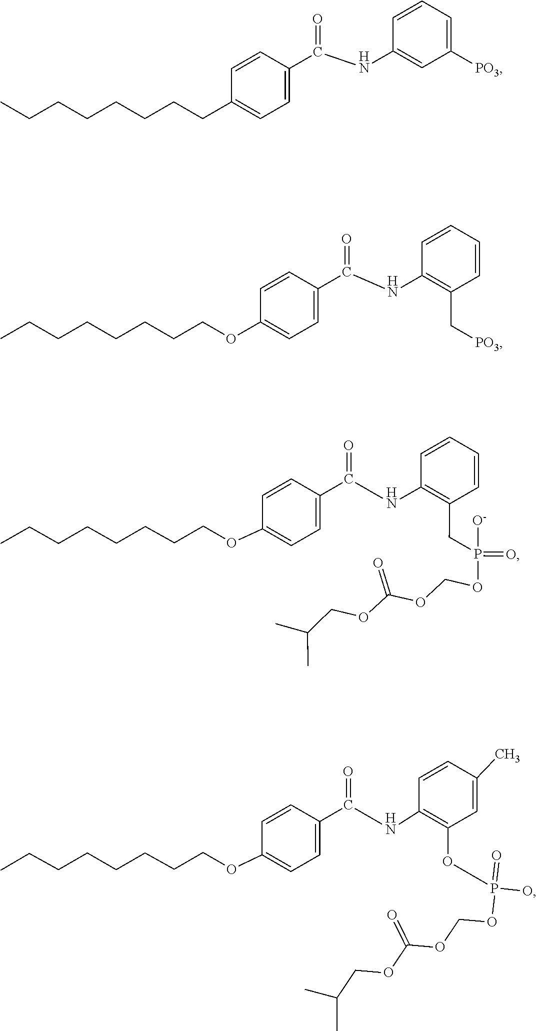

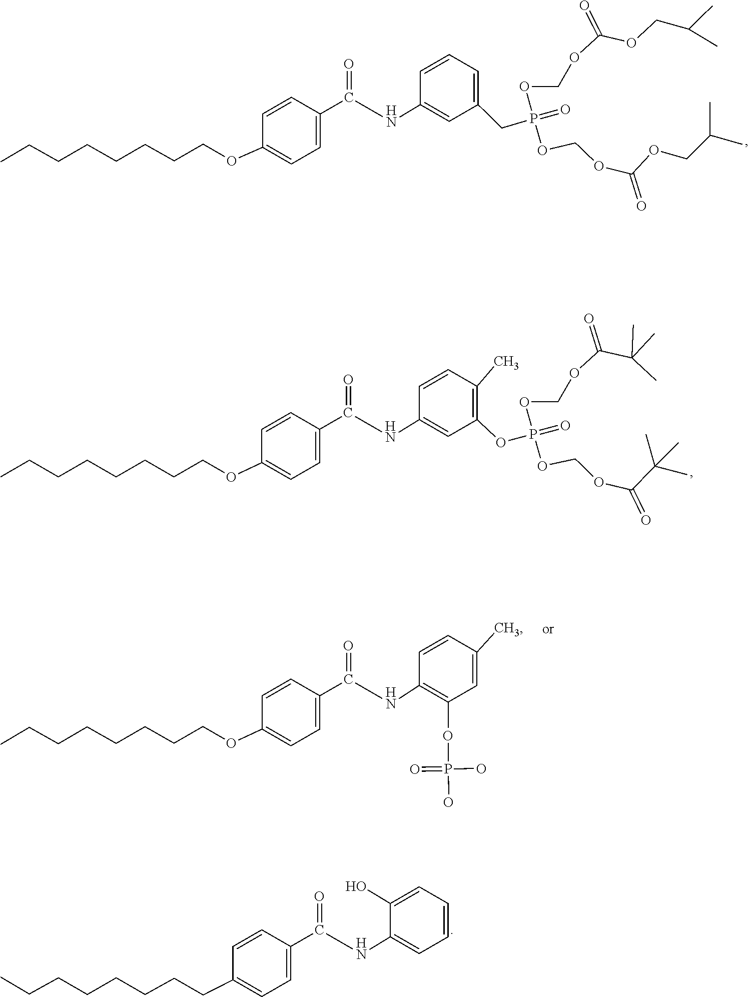

4. The method of claim 1, wherein the compound or its pharmaceutically acceptable salt has the structure of:

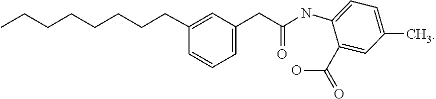

5. The method of claim 1, wherein the compound or its pharmaceutically acceptable salt has the structure of one or more of:

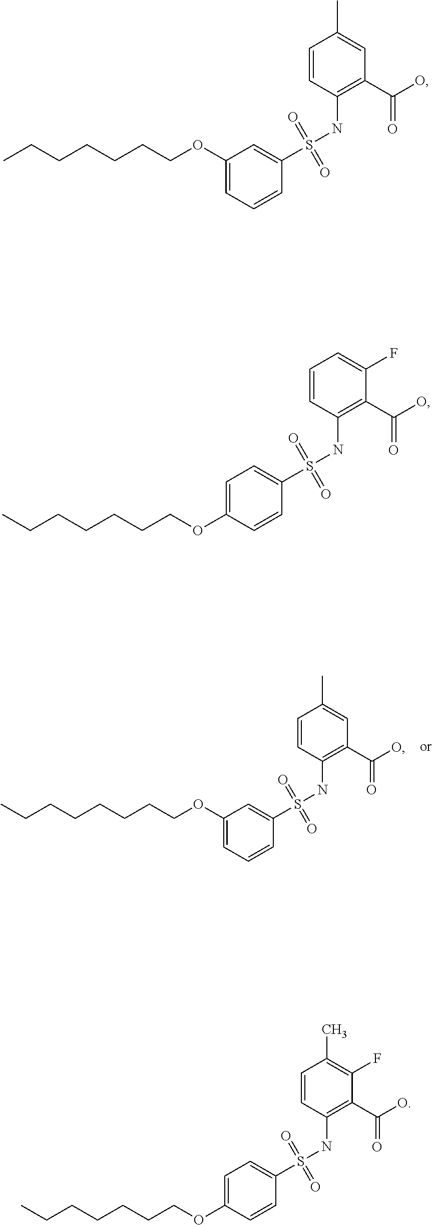

6. The method of claim 1, wherein the cancer is selected from a group consisting of breast cancer, colon cancer, CNS, leukemia, melanoma, prostate, or renal cancer.

7. The method of claim 1, wherein the compound is administered at a dosage of from about 20 mg/kg to about 200 mg/kg.

8. The method of claim 1, wherein the compound is administered at a dosage of from about 35 mg/kg to about 175 mg/kg.

9. The method of claim 1, wherein the compound is administered orally or intratumorally.

10. The method of claim 1, wherein the compound is administered daily for about 2 days to about 35 days.

11. The method of claim 1, wherein the compound is administered daily for about 7 days to about 28 days.

12. A method for inhibiting Polo-like Kinase proteins, the method comprising:
providing a small molecule PLK inhibitor to a medium containing one or more Polo-like Kinase proteins, wherein the small molecule PLK inhibitor has the general structure: 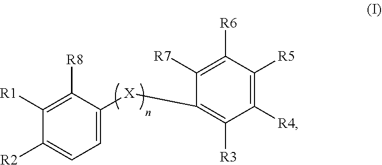
or a pharmaceutically acceptable salt or stereoisomer thereof, wherein:
R1 comprises a hydrogen, —C1 to C8 alkyl, —C1 to C8 alkoxy, or —OH;
R2 is independently selected from hydrogen, (C1 to C8) alkyl, (C1 to C8) alkoxy, secondary (C1 to C8) alkyl methylamine (C8N(Me)), —CONH—(C1 to C6 alkyl), (C1 to C9 alkyl)-sulfinylamino chloride, or NH₂;
R3 through R8 are independently selected from carboxyl, hydrogen, alkyl phosphonate, phosphonate, alkyl phosphonate monoester, phosphate, halogen, alkyl phosphonate disopropylester, phosphate di-t-butylester, —SO₂F, —OSO₂F, —NSO₂F, di-tert-butyl N,N-diethylphosphoramidite, methyl pivalate, or isobutyl methyl carbonates;
X is carbonyl or sulfonyl,
n is 0, 1, or 2.

13. The method of claim 12, wherein the medium comprises a solution.

14. The method of claim 12, wherein the medium comprises one or more cells.

15. The method of claim 14, wherein the one or more cells comprises non-small cell lung cancer (NSCLC) cells, melanoma cells, colon cancer cells, central nervous system (CNS) cancer cells, prostate cancer cells, renal cancer cells, breast cancer cells, or a combination thereof.

16. The method of claim 13, wherein the one or more cells comprises HOP-92, LOX IMVI, HCT-15, MOLT-4, NCI-H460, RPMI-8226, CCRF-CEM, HCT-116, K-562, SR, A549/ATCC, SNB-19, U251, KM12, PC-3, SF-295, NCI-H522, ACHN, SK-MEL-2, MDA-MD-468, SK-MEL-5, UACC-62, UO-31, HL-60(TB), T-47D, MCF7, EKVX, NCI-H23, NIC-H266, or a combination thereof.

17. The method of claim 12, wherein the compound or its pharmaceutically acceptable salt has the structure of one or more of:

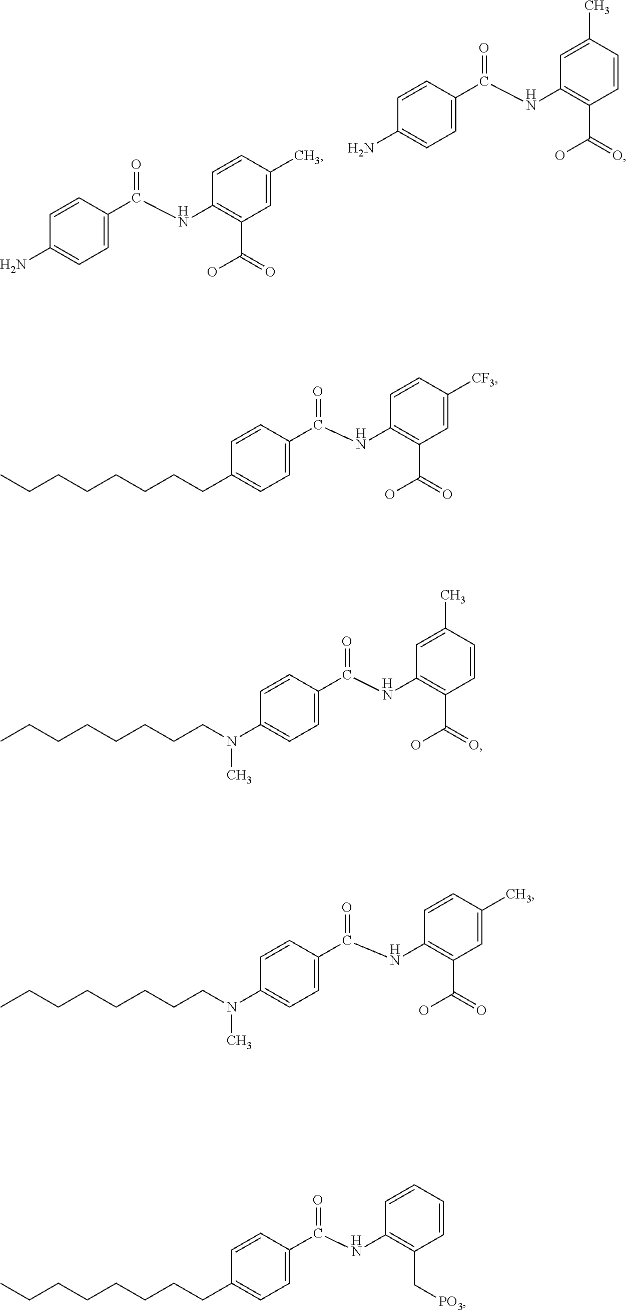

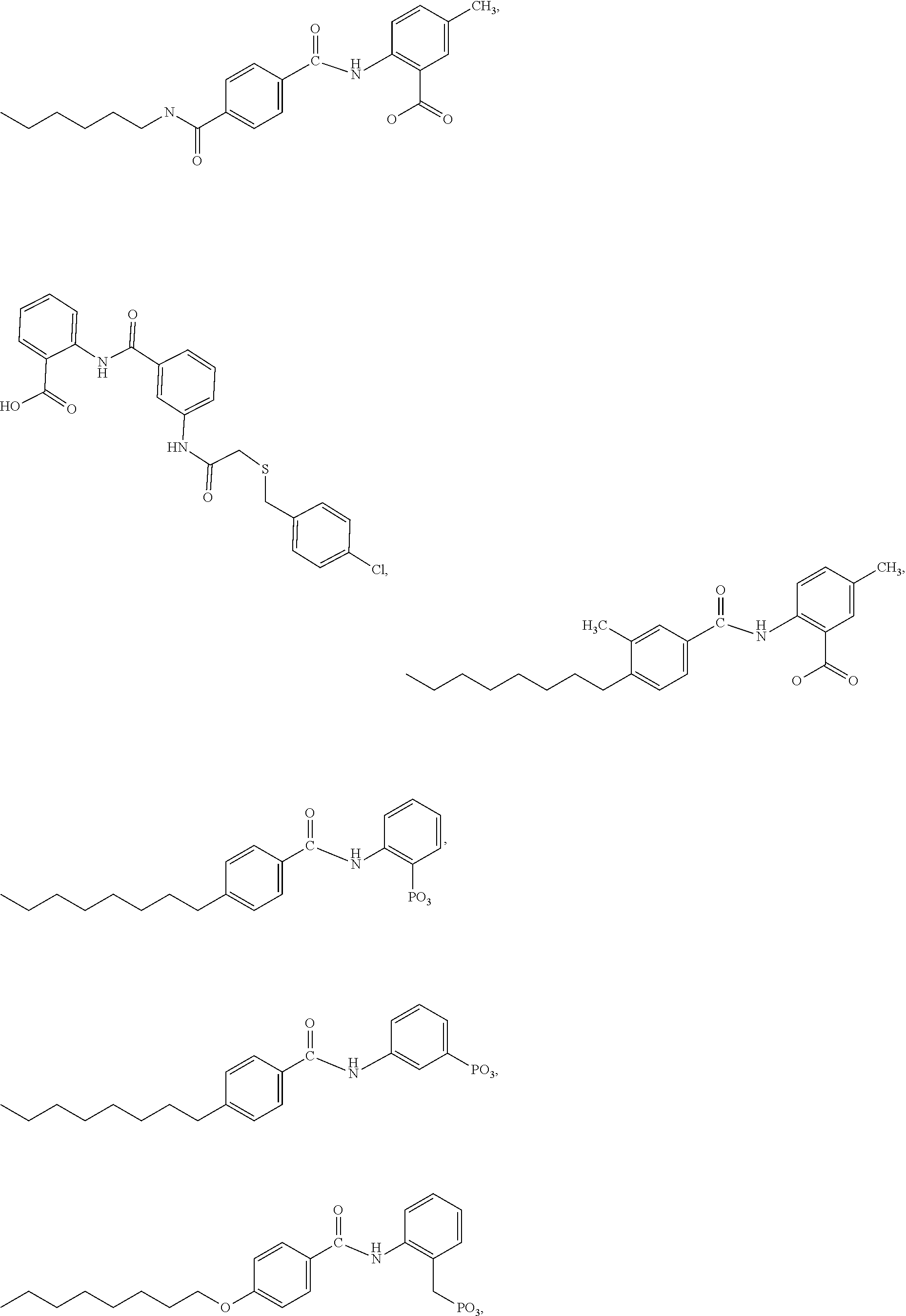

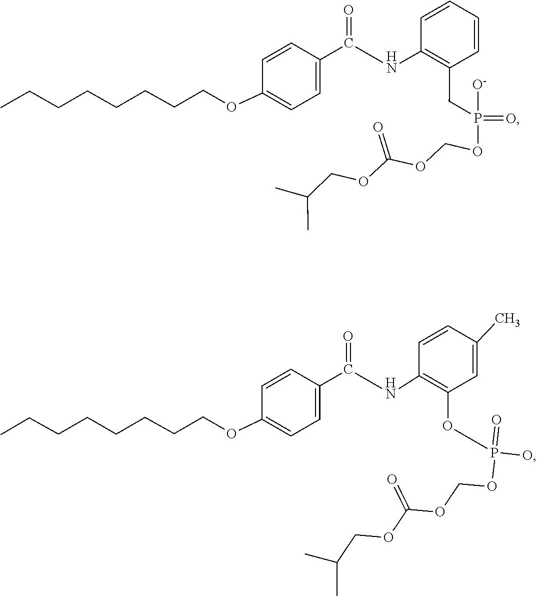

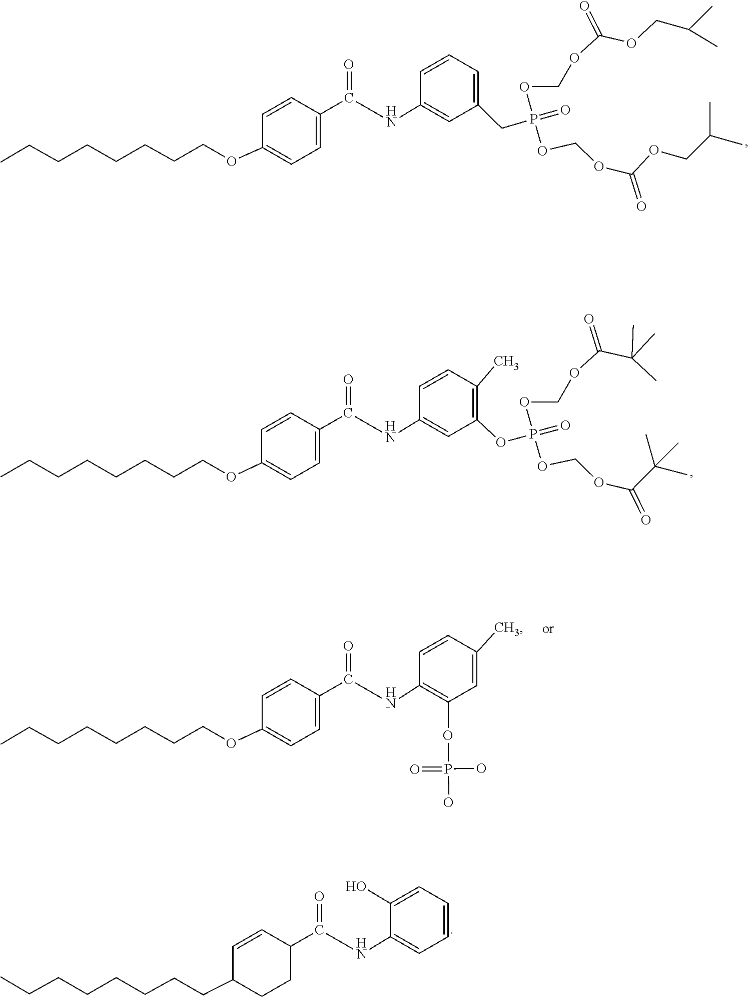

18. The method of claim 12, wherein the compound or its pharmaceutically acceptable salt has the structure of:

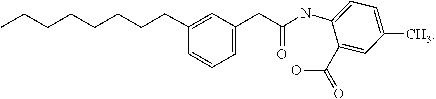

19. The method of claim 12, wherein the compound or its pharmaceutically acceptable salt has the structure of one or more of:

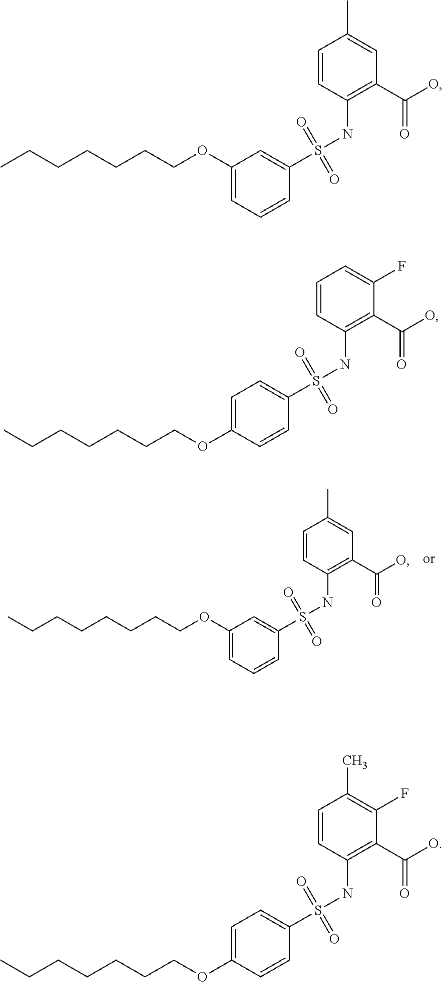

20. The method of claim 12, wherein the small molecule PLK inhibitor has an atomic mass of about 1000 Daltons or less.

21. The method of claim 12, wherein the medium comprises one or more cells and the inhibitor induces apoptosis in at least a portion of the one or more cells.

22. A PLK inhibitor, wherein the PLK inhibit comprising the general structure of: 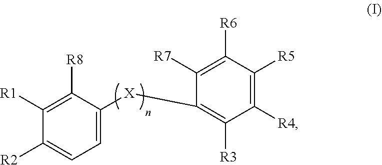
or a pharmaceutically acceptable salt or stereoisomer thereof, wherein:
R1 comprises a hydrogen, —C1 to C8 alkyl, —C1 to C8 alkoxy, or —OH;
R2 is independently selected from hydrogen, (C1 to C8) alkyl, (C1 to C8) alkoxy, secondary (C1 to C8) alkyl methylamine (C8N(Me)), —CONH—(C1 to C6 alkyl), (C1 to C9 alkyl)-sulfinylamino chloride, or NH₂;
R3 through R8 are independently selected from carboxyl, hydrogen, alkyl phosphonate, phosphonate, alkyl phosphonate monoester, phosphate, halogen, alkyl phosphonate disopropylester, phosphate di-t-butylester, —SO₂F, —OSO₂F, —NSO₂F, di-tert-butyl N,N-diethylphosphoramidite, methyl pivalate, or isobutyl methyl carbonates;
X is carbonyl, sulfonyl, amide, —C1 to C4 alkyl-NH—, —NH—, —C1 to C4 alkyl-S—, —SONH—, or —SO₂N—;
n is 0, 1, or 2.

23. A method of inducing degradation of PLK1, the method comprising administering to a subject in need thereof a therapeutically effective amount of a compound of claim 1, or a pharmaceutically acceptable salt thereof.
